(19) United States
(12) Patent Application Publication
(10) Pub. No.: US 2025/0304694 A1
(43) Pub. Date: Oct. 2, 2025

(54) GLYCOSYLATED FC VARIANTS OF WHICH BINDING AFFINITY FOR HUMAN FCGRS IS REMOVED

(71) Applicant: Korea University Research and Business Foundation, Seoul (KR)

(72) Inventors: Sang Taek JUNG, Seoul (KR); Suyeon KIM, Seoul (KR); Migyeong JO, Seoul (KR)

(73) Assignee: Korea University Research and Business Foundation, Seoul (KR)

(21) Appl. No.: 18/864,021

(22) PCT Filed: Jun. 16, 2023
(86) PCT No.: PCT/KR2023/008394

§ 371 Date: Nov. 8, 2024

(30) Foreign Application Priority Data
Jun. 29, 2022 (KR) 10-2022-0079825

(51) Int. Cl. C07K 16/28; A61K 47/68

(52) U.S. Cl. CPC C07K 16/2818; A61K 47/6835

(57) ABSTRACT

The present invention relates to glycosylated FC variants of which binding affinity for human FcγRs is removed, to minimize off-target toxicity of an antibody against an antigen. Novel human antibody Fc domain variants of the present invention, which were discovered using Chinese hamster ovary (CHO) cells having a very similar sugar profile to humans, have significantly reduced binding to Fc gamma receptors, compared to wild-type human antibody Fc domain and conventional S228P or S228P/L235E variants, and are variants of which pH-dependent binding affinity for FcRn and thermal stability are maintained and binding affinity for all FcγRs is completely removed. Therefore, the variants can be used to reduce the toxicity and enhance the efficacy of therapeutic protein drugs and to maintain the half-life of diagnostic/research substances and remove target toxicity.

### TECHNICAL FIELD

The present disclosure relates to a technology capable of minimizing off-target toxicity of an antibody against a target antigen, and to glycosylated Fc variants in which binding affinity for human FcγRs is removed.

### BACKGROUND ART

Protein therapeutic agents have very high specificity for disease targets and low side effects and toxicity and thus are widely used in clinical trials by rapidly replacing non-specific small molecule compound therapeutic agents, and among protein therapeutic agents currently used in clinical trials, antibody therapeutic agents and Fc-fusion protein therapeutic agents that are fused with the Fc domain of an antibody make up the largest part. Therapeutic antibodies show very high specificity for targets compared to conventional small molecule drugs, have not only low biotoxicity and few side effects, but also an excellent blood half-life of about 3 weeks, and thus are considered as one of the most effective cancer therapy methods. In fact, large pharmaceutical companies and research institutes around the world are accelerating the research and development of therapeutic antibodies that specifically bind to cancer cells, including cancer-causing factors, to effectively remove the cancer cells. Companies for developing therapeutic antibody drugs mainly consist of pharmaceutical companies such as Roche, Amgen, Johnson & Johnson, Abbott, and BMS. Particularly, Roche includes representative products of Herceptin, Avastin, Rituxan, and the like for anti-cancer treatment, and these three therapeutic antibodies not only achieved large profits, achieving sales of approximately $19.5 billion in the global market in 2012, but are also leading the antibody drug market of the world. Johnson & Johnson, which developed Remicade, is also growing rapidly in the global antibody market due to increased sales, and pharmaceutical companies such as Abbott and BMS are also known to have many therapeutic antibodies in the final stages of development. As a result, biopharmaceuticals containing therapeutic antibodies that are specific for disease targets and have low side effects are rapidly replaced in the global pharmaceutical market, where small molecule drugs had the initiative. The antibody provides a linkage between the humoral and cellular immune systems, and a Fab region of the antibody recognizes an antigen, whereas an Fc domain region binds to a receptor (Fc receptor or FcR) for an antibody (immunoglobulin) on a cell that is differentially expressed by all immune competent cells, and it has different mechanisms depending on the type of FcγR expressed on the surface of the binding immune cell. An Fc receptor binding site on the Fc domain of the antibody binds to the Fc receptor (FcR) on the cell, and the antibody binds to the Fc receptor on the cell surface through the Fc domain to trigger a variety of important biological responses, including control of phagocytosis and destruction of antibody-coated particles, removal of immune complexes, lysis of antibody-coated target cells by killing cells (antibody-dependent cell-mediated cytotoxicity, or ADCC), release of inflammatory mediators, placental transfer and immunoglobulin production (Deo, Y. M. et al., Immunol. Today 18 (3): 127-135 (1997)). As such, the Fc domain plays a critical role in the collection of immune cells and antibody-dependent cell-mediated cytotoxicity (ADCC) and antibody dependent cell-mediated phagocytosis (ADCP). In particular, the ADCC and ADCP functions, which are the effector functions of the antibody, depend on interaction with Fc receptors present on the surfaces of many cells. Human Fc receptors are classified into five types, and the type of immune cell to be collected is determined depending on which Fc receptor the antibody binds to. For example, the Fc domain of the antibody is responsible for the major therapeutic effect of therapeutic antibodies by inducing the effector functions of ADCC by binding to FcγRIIIa, ADCP by binding to FcγRI or FcγRIIa, and complement dependent cytotoxicity (CDC) by binding to C1q to have toxicity to the target antigen binding to the Fab region.

However, in the therapeutic context, the effector functions of the antibody are often undesirable and may cause safety problems and unwanted side effects by activating host immune defenses. For example, some therapeutic antibodies, such as immune checkpoint inhibitors that bind to immune cells and bispecific immune cell engagers, have had a problem of side effects in which the immune action mechanism is shown in the targeted immune cells to destroy the immune cells. Immune checkpoint inhibitors that target immune checkpoint proteins expressed on the surface of immune cells such as T cells have the disadvantage of lowering the original effects of antibodies by causing the side effects of destroying immune cells that should remove cancer cells by activating the immune responses due to an Fc-mediated immune action mechanism. In addition, in the bispecific immune cell engager which is an antibody therapeutic agent that acts to more effectively remove the cancer cells by guiding immune cells to cancer cells with the antibody therapeutic agent of which one side binds to an antigen of the surface of the cancer cell and the other side binds to the immune cell, when the corresponding antibody has an Fc-mediated immune mechanism, the immune cells are destroyed and the cancer cells are not effectively removed, resulting in side effects. In addition, there is a problem that agonist antibodies that bind to target cells to induce cell activation, or antagonist antibodies that block the interaction between the target antigen and the ligand, have toxicity to target cells and antigens due to the Fc-mediated immune mechanism, thereby reducing the original effects of the antibodies. In addition, when developing an Fc-fusion protein fused with the Fc domain to increase the half-life of active substances such as proteins or chemicals for therapeutic, diagnostic, or research purposes, there is a problem of toxicity due to the Fc-mediated immune mechanism.

Therefore, in order to prevent off-target toxicity of an antibody due to the immune mechanism of the antibody and have effective antibody therapeutic effects, it is essential to remove the Fc-mediated immune mechanism. To this end, when developing antibodies, IgG2 antibodies, which have a very low immune mechanism due to the lowest binding affinity for FcγR among human IgG subclasses, are considered. However, the IgG2 antibodies have various allotypes due to disulfide bond exchange in the hinge region, and have physical property problems that cause aggregation due to decreased stability, and thus IgG4 antibodies having the next lowest binding affinity are considered and then currently used in clinical development. In this regard, anti-PD-1 antibodies (pembrolizumab (Keytruda) from Merck & Co., nivolumab (Opdivo) from Bristol-Myers Squibb, and cemiplimab (Libtayo) from Regeneron) targeting PD-1 (Programmed cell death-1) as an immune checkpoint protein expressed on T cells are all human IgG4 antibodies that have received FDA approval and have been used in high demand in clinical trials. The pembrolizumab has received clinical approval for various types of cancer and ranked second in global pharmaceutical sales in 2020, with sales of $14.3 billion, and the nivolumab ranked eighth, with sales of $7.9 billion. However, the IgG4 antibodies also have binding affinity for all FcγRs, and in particular, have strong binding affinity of several nM for FcγRI, and thus have a problem of causing activation of various immune mechanisms. Therefore, Fc with removed binding affinity for FcγRs is required to prevent target cells from being destroyed by the immune mechanism of the antibody.

### DISCLOSURE

### Technical Problem

An object of the present disclosure is to provide a novel human antibody Fc domain variant.

Another object of the present disclosure is to provide an antibody with a reduced effector function or an immunologically active fragment thereof.

Yet another object of the present disclosure is to provide an antibody therapeutic agent.

Still another object of the present disclosure is to provide a pharmaceutical composition for treating or preventing cancer.

Still another object of the present disclosure is to provide a method for preparing a human antibody Fc domain variant.

Still another object of the present disclosure is to provide a method for preparing an antibody with a reduced effector function.

Still another object of the present disclosure is to provide a use of the Fc domain variant, the antibody or the immunologically active fragment thereof for the preparation of the antibody therapeutic agent.

Still another object of the present disclosure is to provide a use for preventing or treating cancer.

Still another object of the present disclosure is to provide a method for treating cancer.

### Technical Solution

In order to solve the problems, an aspect of the present disclosure provides a novel human antibody Fc domain variant with a reduced effector function.

Another aspect of the present disclosure provides an antibody or an immunologically active fragment thereof including the novel human antibody Fc domain variant.

Yet another aspect of the present disclosure provides an antibody therapeutic agent in which the antibody or immunologically active fragment thereof is conjugated with a therapeutic agent.

Still another aspect of the present disclosure provides a pharmaceutical composition for treating or preventing cancer comprising the Fc domain variant, the antibody or immunologically active fragment thereof, or the antibody therapeutic agent as an active ingredient.

Still another aspect of the present disclosure provides a method for preparing the human antibody Fc domain variant.

Still another aspect of the present disclosure provides a method for preparing an antibody with a reduced effector function.

Still another object of the present disclosure is to provide a use of the Fc domain variant, the antibody or the immunologically active fragment thereof for the preparation of the antibody therapeutic agent.

Still another aspect of the present disclosure provides a use of treating or preventing cancer of the Fc domain variant, the antibody or the immunologically active fragment thereof, or the antibody therapeutic agent.

Still another aspect of the present disclosure provides a method for treating cancer comprising administering the Fc domain variant, the antibody or the immunologically active fragment thereof, or the antibody therapeutic agent in a pharmaceutically effective amount to a subject with cancer.

### Advantageous Effects

According to the present disclosure, novel human antibody Fc domain variants, which were discovered using Chinese hamster ovary (CHO) cells having a very similar sugar profile to humans, have significantly reduced binding to Fc gamma receptors, compared to wild-type human antibody Fc domain and conventional S228P or S228P/L235E variants, and are variants of which pH-dependent binding affinity for FcRn and thermal stability are maintained and binding affinity for all FcγRs is completely removed. Therefore, the variants can be used to reduce the toxicity and enhance the efficacy of therapeutic protein drugs and to maintain the half-life of diagnostic/research substances and remove target toxicity.

### DESCRIPTION OF DRAWINGS

FIG. 1 is a schematic diagram of a mammalian cell display technology for glycosylated Fc.

FIG. 2 is a diagram illustrating results of SDS-PAGE analysis after expression and purification of purified tetrameric FcRI-streptavidin, tetrameric FcγRIIIa-158V-streptavidin, FcγRI-GST, FcγRIIa-131H-GST, FcγRIIa-131R-GST, FcγRIIb-GST, FcγRIIIa-158V-GST, FcγRIIIa-158F-GST, and FcRn-GST.

FIG. 3 illustrates results of analyzing the binding activity of fluorescently-labeled tetrameric FcγRI-Alexa647, tetrameric FcγRIIIa-Alexa647, and Protein A-FITC to wild-type Fc expressed in CHO cells.

FIG. 4 is a schematic diagram of a site-directed mutation library for glycosylated Fc engineering.

FIG. 5 is a schematic diagram of glycosylated Fc engineering using a CHO cell display and a result showing the frequency of mutations in glycosylated Fc variants obtained through screening.

FIG. 6 is a diagram illustrating results of SDS-PAGE analysis after purification of sorted glycosylated Fc variants SL001, SL002, SL003, SL004, SL005, SL006, SL007, and SL008.

FIG. 7 illustrates results of ELISA analysis of binding affinity for FcγRI and FcγRIIIa-158V of glycosylated pembrolizumab Fc variants introduced with the sorted glycosylated Fc variants SL001, SL002, SL003, SL004, SL005, SL006, SL007, and SL008, respectively.

FIG. 8 illustrates results of SDS-PAGE analysis after purifying pembrolizumab Fc variants SPEC, SPFG and SPECFG including recombinant glycosylated Fc variants that prevent Fab-arm exchange, respectively.

FIG. 9 illustrates results of ELISA analysis of binding affinity for FcγRI, FcγRIIa-131H, FcγRIIa-131R, FcγRIIb, FcγRIIIa-158V and FcγRIIIa-158F of recombinant glycosylated pembrolizumab Fc variants SPEC, SPFG and SPECFG that prevent Fab-arm exchange.

FIG. 10 illustrates results of ELISA analysis of binding affinity for C1q of recombinant glycosylated pembrolizumab Fc variants SPEC, SPFG and SPECFG that prevent Fab-arm exchange.

FIG. 11 illustrates results of ELISA analysis of binding affinity for FcRn of recombinant glycosylated pembrolizumab Fc variants SPEC, SPFG and SPECFG that prevent Fab-arm exchange.

FIG. 12 illustrates results of DSF analysis of thermal stability of recombinant glycosylated pembrolizumab Fc variants SPEC, SPFG and SPECFG that prevent Fab-arm exchange.

FIG. 13 illustrates results of SDS-PAGE analysis after purifying glycosylated Fc variants EC and FG sorted in the present disclosure, a combined glycosylated Fc variant ECFG thereof, and recombinant glycosylated Fc variants SPEC, SPFG, and SPECFG.

FIG. 14 illustrates results of ELISA analysis of binding affinity for various human FcγRs of glycosylated pembrolizumab Fc variants EC, FG, ECFG, SPEC, SPFG, and SPECFG of the present disclosure.

FIG. 15 illustrates results of ELISA analysis of binding affinity for C1q of glycosylated pembrolizumab Fc variants EC, FG, ECFG, SPEC, SPFG, and SPECFG of the present disclosure.

FIG. 16 illustrates results of ELISA analysis of binding affinity for FcRn of glycosylated pembrolizumab Fc variants EC, FG, ECFG, SPEC, SPFG, and SPECFG of the present disclosure.

FIG. 17 illustrates results of DSF analysis of thermal stability of glycosylated pembrolizumab Fc variants EC, FG, ECFG, SPEC, SPFG, and SPECFG of the present disclosure.

FIG. 18 is a diagram illustrating results of SDS-PAGE analysis after expression and purification of purified murine FcγRI-GST, murine FcγRIIb-GST, murine FcγRIII-GST, murine FcγRIV-GST, cynomolgus monkey FcγRI-GST, cynomolgus monkey FcγRIIa-GST, cynomolgus monkey FcγRIIb-GST, and cynomolgus monkey FcγRIII-GST.

FIG. 19 illustrates results of ELISA analysis of binding affinity for various murine FcγRs of glycosylated pembrolizumab Fc variants EC, FG, ECFG, SPEC, SPFG, and SPECFG of the present disclosure.

FIG. 20 illustrates results of ELISA analysis of binding affinity for various cynomolgus monkey FcγRs of glycosylated pembrolizumab Fc variants EC, FG, ECFG, SPEC, SPFG, and SPECFG of the present disclosure.

### BEST MODE OF THE INVENTION

Hereinafter, exemplary embodiments of the present disclosure will be described in detail with reference to the accompanying drawings. However, the following exemplary embodiments are presented as examples for the present disclosure, and when it is determined that a detailed description of well-known technologies or configurations known to those skilled in the art may unnecessarily obscure the gist of the present disclosure, the detailed description thereof may be omitted, and the present disclosure is not limited thereto. Various modifications and applications of the present disclosure are possible within the description of claims to be described below and the equivalent scope interpreted therefrom.

Further, terminologies used in the present disclosure are terminologies used to properly express preferred exemplary embodiments of the present disclosure, which may vary according to a user, an operator's intention, or customs in the art to which the present disclosure pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

All technical terms used in the present disclosure, unless otherwise defined, have the meaning as commonly understood by those skilled in the related art of the present disclosure. In addition, although preferred methods and samples are described in the present disclosure, similar or equivalent methods and samples thereto are also included in the scope of the present disclosure. The contents of all publications disclosed as references in this specification are incorporated in the present disclosure.

Throughout the present specification, general one-letter or three-letter codes for naturally existing amino acids are used, and generally allowed three-letter codes for other amino acids, such as α-aminoisobutyric acid (Aib) and N-methylglycine (Sar) are also used. The amino acids mentioned in the present disclosure as abbreviations are also described as follows according to the IUPAC-IUB nomenclature.

Alanine: A: Arginine: R: Asparagine: N: Aspartic acid: D: Cysteine: C: Glutamic acid: E: Glutamine: Q: Glycine: G; Histidine: H: Isoleucine: I; Leucine: L; Lysine: K: Methionine: M: Phenylalanine: F: Proline: P: Serine: S: Threonine: T: Tryptophan: W: Tyrosine: Y; and Valine: V.

In one aspect, the present disclosure relates to a human antibody Fc domain variant in which one or more amino acids selected from the group consisting of amino acids at positions 228, 233, 234, 291, 309 and 402 numbered according to a Kabat numbering system in a wide-type human antibody Fc domain are substituted with sequences different from wild-type amino acids.

In one embodiment, the human antibody Fc domain variant of the present disclosure may include one or more amino acid substitutions selected from the group consisting of S228P, E233C, E233P, E233G, F234G, F234T, F234R, P291S, L309P and G402D.

In one embodiment, the human antibody Fc domain variant of the present disclosure may include amino acid substitutions of E233C and/or F234G.

In one embodiment, the human antibody Fc domain variant of the present disclosure may be a human antibody Fc domain variant SPEC including amino acid substitutions of S228P and E233C, and the human antibody Fc domain variant SPEC may include an amino acid sequence represented by SEQ ID NO: 1, which may be encoded by a nucleic acid molecule including a nucleotide sequence represented by SEQ ID NO: 2.

In one embodiment, the human antibody Fc domain variant of the present disclosure may be a human antibody Fc domain variant SPFG including amino acid substitutions of S228P and F234G, and the human antibody Fc domain variant SPFG may include an amino acid sequence represented by SEQ ID NO: 3, which may be encoded by a nucleic acid molecule including a nucleotide sequence represented by SEQ ID NO: 4.

In one embodiment, the human antibody Fc domain variant of the present disclosure may be a human antibody Fc domain variant ECFG including amino acid substitutions of E233C and F234G, and the human antibody Fc domain variant ECFG may include an amino acid sequence represented by SEQ ID NO: 5, which may be encoded by a nucleic acid molecule including a nucleotide sequence represented by SEQ ID NO: 6.

In one embodiment, the human antibody Fc domain variant of the present disclosure may be a human antibody Fc domain variant SPECFG including amino acid substitutions of S228P, E233C and F234G, and the human antibody Fc domain variant SPECFG may include an amino acid sequence represented by SEQ ID NO: 7, which may be encoded by a nucleic acid molecule including a nucleotide sequence represented by SEQ ID NO: 8.

In one embodiment, the human antibody Fc domain variant of the present disclosure may be a human antibody Fc domain variant SL001 including an amino acid substitution of E233C, and the human antibody Fc domain variant SL001 may include an amino acid sequence represented by SEQ ID NO: 9, which may be encoded by a nucleic acid molecule including a nucleotide sequence represented by SEQ ID NO: 10.

In one embodiment, the human antibody Fc domain variant of the present disclosure may be a human antibody Fc domain variant SL002 including amino acid substitutions of E233P and P291S, and the human antibody Fc domain variant SL002 may include an amino acid sequence represented by SEQ ID NO: 19, which may be encoded by a nucleic acid molecule including a nucleotide sequence represented by SEQ ID NO: 20.

In one embodiment, the human antibody Fc domain variant of the present disclosure may be a human antibody Fc domain variant SL003 including an amino acid substitution of E233G, and the human antibody Fc domain variant SL003 may include an amino acid sequence represented by SEQ ID NO: 21, which may be encoded by a nucleic acid molecule including a nucleotide sequence represented by SEQ ID NO: 22.

In one embodiment, the human antibody Fc domain variant of the present disclosure may be a human antibody Fc domain variant SL004 including amino acid substitutions of E233G and L309P, and the human antibody Fc domain variant SL004 may include an amino acid sequence represented by SEQ ID NO: 23, which may be encoded by a nucleic acid molecule including a nucleotide sequence represented by SEQ ID NO: 24.

In one embodiment, the human antibody Fc domain variant of the present disclosure may be a human antibody Fc domain variant SL006 including amino acid substitutions of F234G and G402D, and the human antibody Fc domain variant SL006 may include an amino acid sequence represented by SEQ ID NO: 25, which may be encoded by a nucleic acid molecule including a nucleotide sequence represented by SEQ ID NO: 26.

In one embodiment, the human antibody Fc domain variant of the present disclosure may be a human antibody Fc domain variant SL007 including an amino acid substitution of F234T, and the human antibody Fc domain variant SL007 may include an amino acid sequence represented by SEQ ID NO: 27, which may be encoded by a nucleic acid molecule including a nucleotide sequence represented by SEQ ID NO: 28.

In one embodiment, the human antibody Fc domain variant of the present disclosure may be a human antibody Fc domain variant SL008 including an amino acid substitution of F234R, and the human antibody Fc domain variant SL008 may include an amino acid sequence represented by SEQ ID NO: 29, which may be encoded by a nucleic acid molecule including a nucleotide sequence represented by SEQ ID NO: 30.

In one embodiment, the human antibody Fc domain variant of the present disclosure may be a human antibody Fc domain variant SL005 including an amino acid substitution of F234G, and the human antibody Fc domain variant SL005 may include an amino acid sequence represented by SEQ ID NO: 11, which may be encoded by a nucleic acid molecule including a nucleotide sequence represented by SEQ ID NO: 12.

In one embodiment, the human antibody (immunoglobulin) may be IgA, IgM, IgE, IgD or IgG, or modifications thereof, and may be IgG1, IgG2, IgG3 or IgG4, more preferably IgG4, more preferably an anti-PD antibody, and pembrolizumab.

In one embodiment, the human antibody (immunoglobulin) may be IgG4 or a modification thereof, and the Fc domain of wild-type IgG4 including hinge, CH2 and CH3 may include an amino acid sequence represented by SEQ ID NO: 13 and be encoded by a nucleic acid molecule including a nucleotide sequence represented by SEQ ID NO: 14.

In one embodiment, in a human antibody IgG4 Fc domain, or an IgG4 Fc domain variant including an amino acid substitution of conventional S228P, the amino acids at positions 233 and/or 234 numbered according to the Kabat numbering system in the variants of the present disclosure may be additionally substituted with sequences different from amino acids of the wild type.

In one embodiment, the IgG4 Fc domain variant including the amino acid substitution of S228P may include an amino acid sequence represented by SEQ ID NO: 15 and be encoded by a nucleic acid molecule including a nucleotide sequence represented by SEQ ID NO: 16.

In one embodiment, the IgG4 Fc domain variant including the amino acid substitutions of S228P and L235E may include an amino acid sequence represented by SEQ ID NO: 17 and be encoded by a nucleic acid molecule including a nucleotide sequence represented by SEQ ID NO: 18.

In one embodiment, the human antibody Fc domain variant of the invention may have reduced binding affinity for Fc gamma receptors (FcγRs) compared to the wild-type human antibody Fc domain, and the Fc gamma receptors may be FcγRI, FcγRIIa, FcγRIIb or FcγRIIIa.

In one embodiment, the human antibody Fc domain variant of the present disclosure may have reduced binding affinity for C1q compared to the wild-type human antibody Fc domain.

In one embodiment, the human antibody Fc domain variant of the present disclosure may have a reduced effector function compared to the wild-type human antibody Fc domain.

In one embodiment, the effector function may be an Fc-mediated effector function selected from C1q-binding, complement activation, complement dependent cytotoxicity (CDC), antibody-dependent cellular cytotoxicity (ADCC), Fc-receptor binding including Fc-gamma receptor binding, protein A-binding, protein G-binding, antibody-dependent cell-mediated phagocytosis (ADCP), complement dependent cell-mediated cytotoxicity (CDCC), complement-enhanced cytotoxicity, opsonization, Fc-containing polypeptide internalization, target downmodulation, ADC uptake, induction of apoptosis, cell death, cell cycle arrest, and any combination thereof, preferably ADCC or CDC.

In one embodiment, the human antibody Fc domain variant of the present disclosure may have similar pH-dependent FcRn binding affinity to that of a wild-type human antibody Fc domain and may have an in vivo half-life and thermal stability similar to that of a wild-type human antibody Fc domain.

In one embodiment, the Fc domain variants of the present disclosure may be used for the purpose of not killing binding cells.

In one embodiment, the Fc domain variants of the present disclosure may be applied to antibodies targeting immune cells or normal cells.

In one embodiment, the Fc domain variants of the present disclosure may be used in an immune checkpoint inhibitor antibody or a bispecific immune cell engaging bispecific antibody.

In the present disclosure, variants including amino acid mutations in the human antibody Fc domain of the present disclosure are defined according to the amino acid modifications constituting an Fc domain of a parent antibody, and conventional antibody numbering is followed by the EU index by Kabat (Kabat et al., Sequence of proteins of immunological interest, 5th Ed., United States Public Health Service, National Institutes of Health, Bethesda, 1991).

As used in the present disclosure, the term “Fc domain variant” may be used interchangeably with the “Fc variant”.

As used in the present disclosure, the term “wild-type polypeptide” refers to an unmodified polypeptide that is modified later to produce a derivative. The wild-type polypeptide may be a polypeptide found in nature, or a derivative or manipulation of the polypeptide found in nature. The wild-type polypeptide may refer to a polypeptide itself, a composition comprising the wild-type polypeptide, or an amino acid sequence encoding the same. Accordingly, as used in the present disclosure, the term “wild-type antibody” refers to an unmodified antibody polypeptide in which amino acid residues are modified to produce a derivative. Interchangeably with the term, the “parent antibody” may be used to refer to an unmodified antibody polypeptide into which amino acid modifications are introduced to produce a derivative.

As used in the present disclosure, the term “amino acid modification/mutation” refers to substitution, insertion and/or deletion, preferably substitution of amino acids in a polypeptide sequence. As used in the present disclosure, the term “amino acid substitution” or “substitution” means that an amino acid at a specific position in the polypeptide sequence of the wild-type human antibody Fc domain is replaced with another amino acid. For example, an Fc variant including S228P substitution means that an amino acid residue at position 228 in the amino acid sequence of the wild-type antibody Fc domain, serine is replaced with proline.

As used in the present disclosure, the term “Fc variant” means including a modification of one or more amino acid residues compared to the wild-type antibody Fc domain.

The Fc variant of the present disclosure includes one or more amino acid modifications compared to the wild-type antibody Fc domain (region or fragment), resulting in a difference in amino acid sequence. The amino acid sequence of the Fc variant according to the present disclosure is substantially homologous to the amino acid sequence of the wild-type antibody Fc domain. For example, the amino acid sequence of the Fc variant according to the present disclosure has about 80% or more, preferably about 90% or more, and most preferably about 95% or more homology compared to the amino acid sequence of the wild-type antibody Fc domain. The amino acid modifications may be performed genetically using molecular biological methods, or also performed using enzymatic or chemical methods.

The Fc variants of the present disclosure may be prepared by any method known in the art. In one embodiment, the human antibody Fc variant according to the present disclosure is used to form a nucleic acid in which a polypeptide sequence including a specific amino acid modification is encoded and then, if desired, cloned into a host cell, expressed, and assayed. Various methods therefor are described in the literature (Molecular Cloning—A Laboratory Manual, 3rd Ed., Maniatis, Cold Spring Harbor Laboratory Press, New York, 2001; Current Protocols in Molecular Biology, John Wiley & Sons).

The nucleic acid encoding the Fc variant according to the present disclosure may be inserted into an expression vector for protein expression. The expression vector generally includes a protein operably linked, i.e., functionally related to a control or regulatory sequence, a selectable marker, an optional fusion partner, and/or an additional element. Under appropriate conditions, the Fc variant according to the present disclosure may be produced by a method of inducing the protein expression by incubating a host cell transformed with nucleic acid, preferably a nucleic acid-containing expression vector encoding the Fc variant according to the present disclosure. A variety of suitable host cells including mammalian cells, bacteria, insect cells, and yeast may be used, but are not limited thereto. Methods for introducing exogenous nucleic acid into a host cell are known in the art and will vary depending on a host cell to be used. Preferably, the Fc variant according to the present disclosure is produced using E. coli, which has low production cost and high industrial value, as a host cell.

Accordingly, the scope of the present disclosure includes a method for preparing an Fc variant comprising incubating a host cell into which a nucleic acid encoding an Fc variant has been introduced, under conditions suitable for protein expression; and purifying or isolating the Fc variant expressed from the host cell.

As used in the present disclosure, the term “FcRn” or “neonatal Fc receptor” refers to a protein that binds to an IgG antibody Fc domain, which is at least partially encoded by an FcRn gene. The FcRn may be derived from any organism, including humans, mice, rats, rabbits, and monkeys, but is not limited thereto. As known in the art, a functional FcRn protein includes two polypeptides, often referred to as light and heavy chains. The light chain is β-2-microglobulin, and the heavy chain is encoded by the FcRn gene. Unless otherwise stated herein, the FcRn or one FcRn protein refers to a complex of the FcRn heavy chain and β-2-microglobulin.

In one aspect, the present disclosure relates to an antibody including the Fc domain variant of the present disclosure or an immunologically active fragment thereof.

In one embodiment, the antibody or immunologically active fragment thereof may have reduced binding affinity for Fc gamma receptors (FcγRs) or C1q compared to a wild-type human antibody, because the binding affinity for FcγRs is removed.

In one embodiment, the Fc gamma receptor (FcγR) may be FcγRI, FcγRIIa, FcγRIIb or FcγRIIIa, and may be human FcγR, murine FcγR or monkey FcγR.

In one embodiment, the human FcγR may be FcγRI, FcγRIIa, FcγRIIb or FcγRIIIa, in which the FcγRIIIa may be FcγRIIIa-158V or FcγRIIIa-158F; the murine FcγR may be murine FcγRI, murine FcγRIIIb, murine FcγRIII or murine FcγIV; and the monkey FcγR may be cynomolgus monkey FcγRI, monkey FcγRIIa, monkey FcγRIIb or monkey FcγRIII.

In one embodiment, the antibody or immunologically active fragment thereof may have a reduced effector function compared to the wild-type human antibody.

In one embodiment, the antibody may be a polyclonal antibody, a monoclonal antibody, a minibody, a domain antibody, a bispecific antibody, an antibody mimetic, a chimeric antibody, an antibody conjugate, a human antibody, or a humanized antibody. The immunologically active fragment may be Fab, Fd, Fab′, dAb, F(ab′), F(ab)2, a single chain fragment variable (scFv), Fv, a single chain antibody, an Fv dimer, a complementarity determining region fragment, or a diabody.

The antibody may be isolated or purified by various methods known in the art. A standard purification method includes chromatographic techniques, electrophoresis, immunology, precipitation, dialysis, filtration, concentration, and chromatofocusing techniques. As known in the art, various natural proteins such as bacterial proteins A, G, and L are bound to the antibody and these proteins may be used for purification. Often, purification by specific fusion partners may be enabled.

The antibody includes functional fragments of an antibody molecule as well as a whole antibody form. The whole antibody has a structure having two full-length light chains and two full-length heavy chains, and each light chain is linked to the heavy chain by disulfide bonds. The functional fragment of the antibody molecule refers to a fragment having an antigen-binding function. Examples of the antibody fragment include (i) a Fab fragment consisting of a light chain variable region VL, a heavy chain variable region VH, a light chain constant region CL, and a first heavy chain constant region CH1: (ii) a Fd fragment consisting of VH and CH1 domains; (iii) an Fv fragment consisting of VL and VH domains of a single antibody: (iv) a dAb fragment consisting of a VH domain (Ward E S et al., Nature 341:544-546 (1989)): (v) an isolated CDR region: (vi) a F(ab)2 fragment, which is a bivalent fragment including two linked Fab fragments: (vii) a single-chain Fv molecule (scFv) in which the VH domain and the VL domain are linked to each other by a peptide linker to form an antigen-binding domain: (viii) a bispecific single-chain Fv dimer (PCT/US92/09965); and (ix) a diabody which is a multivalent or multispecific fragment produced by gene fusion (WO94/13804).

The antibody or the immunologically active fragment thereof of the present disclosure may be selected from the group consisting of animal-derived antibodies, chimeric antibodies, humanized antibodies, human antibodies, and immunologically active fragments thereof. The antibody may be produced recombinantly or synthetically.

The antibody or the immunologically active fragment thereof may be isolated from a living body (not present in a living body) or may non-naturally occur, and for example, may be synthetically or recombinantly produced.

As used in the present disclosure, the “antibody” refers to a substance produced by stimulation of an antigen in the immune system, and the type thereof is not particularly limited, and may be obtained naturally or non-naturally (e.g., synthetically or recombinantly). The antibody is advantageous for mass expression and production because of being very stable in vivo as well as ex vivo and having a long half-life. In addition, since the antibody essentially has a dimer structure, avidity is very high. An intact antibody has a structure having two full-length light chains and two full-length heavy chains, and each light chain is linked to the heavy chain by disulfide bonds. The constant region of the antibody is divided into a heavy chain constant region and a light chain constant region, and the heavy chain constant region has gamma (Y), mu (μ), alpha (α), delta (δ), and epsilon (ε) types, and subclasses include gamma 1 (γ1), gamma 2 (γ2), gamma 3 (γ3), gamma 4 (γ4), alpha 1 (α1) and alpha 2 (α2). The light chain constant region has kappa (κ) and lambda (λ) types.

As used in the present disclosure, the term “heavy chain” is interpreted as meaning including all of a full-length heavy chain including a variable region domain V_{H} including an amino acid sequence having a sufficient variable region sequence to impart specificity to an antigen, three constant region domains C_{H}1, C_{H}2 and C_{H}3 and hinges, and fragments thereof. In addition, the term “light chain” is interpreted as meaning including all of a full-length light chain including a variable region domain V_{L} including an amino acid sequence having a sufficient variable region sequence to impart specificity to an antigen and a constant region domain C_{L}, and fragments thereof.

As used in the present disclosure, the term “Fc domain”, “Fc fragment”, or “Fc region” forms an antibody with a Fab domain/fragment, and the Fab domain/fragment consists of a light chain variable region V_{L} and a heavy chain variable region V_{H}, a light chain constant region C_{L}, and a first heavy chain constant region C_{H}1, and the Fc domain/fragment consists of a second constant region C_{H}2 and a third constant region C_{H}3 of the heavy chain.

In one aspect, the present disclosure relates to a nucleic acid molecule encoding the Fc domain variant of the present disclosure, or an antibody including the Fc domain variant or an immunologically active fragment thereof.

In one aspect, the present disclosure relates to a vector including the nucleic acid molecule and a host cell including the vector.

The nucleic acid molecule of the present disclosure may be isolated or recombinant, and includes not only DNA and RNA in single-stranded and double-stranded forms, but also complementary sequences corresponding thereto. The isolated nucleic acid is a nucleic acid that is isolated from surrounding genetic sequences present in the genome of a subject from which the nucleic acid is isolated, in the case of a nucleic acid isolated from a naturally occurring source. In the case of a nucleic acid synthesized enzymatically or chemically from a template, such as a PCR product, a cDNA molecule, or an oligonucleotide, the nucleic acid produced from such a procedure may be understood as an isolated nucleic acid molecule. The isolated nucleic acid molecule refers to a nucleic acid molecule in the form of a separate fragment or as a component of a larger nucleic acid construct. The nucleic acid is operably linked when disposed in a functional relationship with another nucleic acid sequence. For example, DNA of a pre-sequence or secretory leader is expressed as a preprotein in the form before the polypeptide is secreted to be operably linked to DNA of the polypeptide, and a promoter or enhancer is operably linked to a coding sequence when affecting the transcription of the polypeptide sequence, or a ribosome binding domain is operably linked to a coding sequence when disposed to promote the translation. In general, the operably linked means that DNA sequences to be linked are located contiguously, and that the secretory leader exists contiguously in the same leading frame. However, the enhancer needs not to be contiguously located. The linkage is achieved by ligation at a convenient restriction enzyme site. When there is no site, synthetic oligonucleotide adapters or linkers are used according to conventional methods.

In the isolated nucleic acid molecule encoding the Fc domain variant of the present disclosure, or the antibody including the Fc domain variant or immunologically active fragment thereof, due to the degeneracy of codons or in consideration of codons preferred in an organism in which the protein is to be expressed, it will be well understood by those skilled in the art that various modifications may be made to a coding region within a range without changing the amino acid sequence of the Fc domain variant of the present disclosure, or the antibody including the Fc domain variant or immunologically active fragment thereof to be expressed from the coding region, various modifications or changes may be made within a range without affecting the expression of the gene even in parts other than the coding region, and such modified genes are also included within the scope of the present disclosure. That is, as long as the nucleic acid molecule of the present disclosure encodes a protein having equivalent activity thereto, one or more nucleobases may be mutated by substitution, deletion, insertion, or a combination thereof, which are also included in the scope of the present disclosure. The sequence of such a nucleic acid molecule may be single- or double-stranded, and may be a DNA molecule or an RNA (mRNA) molecule.

The isolated nucleic acid molecule encoding the Fc domain variant of the present disclosure, or the antibody or immunologically active fragment thereof including the Fc domain variant may be inserted into an expression vector for protein expression. The expression vector generally includes a protein operably linked, i.e., functionally related to a control or regulatory sequence, a selectable marker, an optional fusion partner, and/or an additional element. In appropriate conditions, the Fc domain variant of the present disclosure, or the antibody or immunologically active fragment thereof including the Fc domain variant may be produced by a method for inducing the protein expression by incubating a host cell transformed with a nucleic acid, preferably an expression vector including an isolated nucleic acid molecule encoding the Fc domain variant of the present disclosure, the antibody including the Fc domain variant or the immunologically active fragment thereof. A variety of suitable host cells including mammalian cells, bacteria, insect cells, and yeast may be used, but are not limited thereto. Methods for introducing exogenous nucleic acid into a host cell are known in the art and will vary depending on a host cell to be used. Preferably, it is possible to produce E. coli, which has high industrial value due to low production cost, as a host cell.

The vector of the present disclosure may include a plasmid vector, a cosmid vector, a bacteriophage vector, a viral vector, etc., but is not limited thereto. The suitable vector includes a signal sequence or a leader sequence for membrane targeting or secretion in addition to expression regulatory elements such as a promoter, an operator, an initiation codon, a termination codon, a polyadenylation signal, and an enhancer and may be variously produced according to the purpose. The promoter of the vector may be constitutive or inductive. The signal sequence may use a PhoA signal sequence, an OmpA signal sequence, etc. in the case of Escherichia sp. as a host, an α-amylase signal sequence, a subtilisin signal sequence, etc. in the case of Bacillus sp. as a host, an MFa signal sequence, a SUC2 signal sequence, etc. in the case of yeast as a host, and an insulin signal sequence, an α-interferon signal sequence, an antibody molecule signal sequence, etc. in the case of an animal cell as a host, but is not limited thereto. Further, the vector may include a selective marker for selecting a host cell including a vector and a replicable expression vector includes a replication origin.

As used in the present disclosure, the term “vector” refers to a vehicle into which a nucleic acid sequence may be inserted for introduction into a cell capable of replicating the nucleic acid sequence. The nucleic acid sequence may be exogenous or heterologous. The vector may include plasmids, cosmids, and viruses (e.g., bacteriophages), but is not limited thereto. Those skilled in the art may construct vectors by standard recombinant techniques (Maniatis, et al., Molecular Cloning, A Laboratory Manual, Cold Spring Harbor Press, Cold Spring Harbor, N.Y., 1988; and Ausubel et al., In: Current Protocols in Molecular Biology, John, Wiley & Sons, Inc, NY, 1994 etc.).

In one embodiment, when constructing the vector, expression regulatory sequences such as a promoter, a terminator, and an enhancer, sequences for membrane targeting or secretion, etc. may be appropriately selected according to a type of host cell to produce the Fc domain variant, or the antibody including the Fc domain variant or immunologically active fragment thereof and may be variously combined depending on a purpose.

As used in the present disclosure, the term “expression vector” refers to a vector including a nucleic acid sequence encoding at least a portion of a gene product to be transcribed. In some cases, the RNA molecule is then translated into a protein, a polypeptide, or a peptide. The expression vector may include various regulatory sequences. In addition to regulatory sequences that regulate transcription and translation, vectors and expression vectors may also include nucleic acid sequences that provide other functions.

As used in the present disclosure, the term “host cell” includes eukaryotes and prokaryotes, and refers to any transformable organism capable of replicating the vector or expressing a gene encoded by the vector. The host cell may be transfected or transformed by the vector, which means a process in which an exogenous nucleic acid molecule is delivered or introduced into the host cell.

In one embodiment, the host cell may be bacteria or animal cells, the animal cell line may be a CHO cell, a HEK cell or a NSO cell, and the bacteria may be E. coli.

In one aspect, the present disclosure relates to a fusion protein in which the Fc domain variant of the present disclosure, or the antibody or immunologically active fragment thereof is linked to a cargo molecule.

In one embodiment, the cargo molecule may be a detector, a therapeutic agent, a drug, a peptide, a growth factor, a cytokine, a receptor trap, a chemical compound, a carbohydrate moiety, an enzyme, an antibody or a fragment thereof, a DNA-based molecule, a viral vector, or a cytotoxic agent: at least one liposome or nanocarrier loaded with a detector, a therapeutic agent, a drug, a peptide, an enzyme, an antibody or a fragment thereof, a DNA-based molecule, a viral vector, or a cytotoxic agent: or one or more nanoparticles, nanowires, nanotubes, or quantum dots.

In one embodiment, the fusion protein may be an agonist antibody, an antagonist antibody or an antibody therapeutic agent.

In one aspect, the present disclosure relates to an antibody therapeutic agent in which an antibody of the present disclosure or an immunologically active fragment thereof is conjugated to one or more therapeutic agents.

In one embodiment, the antibody therapeutic agent may be an immune checkpoint inhibitor or a bispecific immune cell engager and may have a reduced effector function.

In one embodiment, the therapeutic agent may be a chimeric antigen receptor (CAR) cell therapy, an oncolytic drug, an immunotherapy agent, a cytotoxic agent, an angiogenesis inhibitor, a kinase inhibitor, a costimulatory molecule blocker, an adhesion molecule blocker, an anti-cytokine agent, an anti-CTLA-4 agent, an anti-PD-1 agent, an anti-PD-L1 agent, an anti-PD-L2 agent, a TNF-α cross-linking agent, a TRAIL cross-linking agent, an anti-CD27 agent, an anti-CD30 agent, an anti-CD40 agent, an anti-4-1BB agent, an anti-GITR agent, an anti-OX40 agent, an anti-TRAILR1 agent, an anti-TRAILR2 agent, tagretin, interferon-alpha, clobetasol, peginterferon, prednisone, romidepsin, bexarotene, methotrexate, triamcinolone cream, anti-chemokine, vorinostat, Gabapentin, cyclosporine, rapamycin, FK506, a detectable marker or reporter, a TNF antagonist, an antirheumatic agent, a muscle relaxant, narcotic, a non-steroidal anti-inflammatory drug (NSAID), analgesic, anesthetic, sedative, local anesthetic, neuromuscular blocker, antibacterial, psoriasis therapeutic agent, corticosteroid, anabolic steroid, erythropoietin, immunization, immunoglobulin, immunosuppressant, growth hormone, hormone replacement drug, radiopharmaceutical, antidepressant, antipsychotic, stimulant, asthma drug, beta agonist, inhaled steroid, epinephrine or analogue thereof, cytokine, cytokine antagonist, PD-1 antagonist, adenosine A2AR antagonist, CD73 inhibitor, CTLA-4 inhibitor, TIM-3 inhibitor, LAG-3 inhibitor, anthracycline, or combinations thereof.

In one aspect, the present disclosure relates to a pharmaceutical composition for preventing or treating cancer comprising the human antibody Fc domain variant of the present disclosure, the antibody including the Fc domain variant or immunologically active fragment thereof, or the antibody therapeutic agent including the antibody or immunologically active fragment thereof as an active ingredient.

In one embodiment, the cancer may be any one selected from the group consisting of brain tumor, melanoma, myeloma, non-small cell lung cancer, oral cancer, liver cancer, stomach cancer, colon cancer, breast cancer, lung cancer, bone cancer, pancreatic cancer, skin cancer, head or neck cancer, cervical cancer, ovarian cancer, colorectal cancer, small intestine cancer, rectal cancer, fallopian tube carcinoma, perianal cancer, endometrial carcinoma, vaginal carcinoma, vulvar carcinoma, Hodgkin's disease, esophageal cancer, lymph adenocarcinoma, bladder cancer, gallbladder cancer, endocrine adenocarcinoma, thyroid cancer, parathyroid cancer, adrenal cancer, soft tissue sarcoma, urethral cancer, penile cancer, prostate cancer, chronic or acute leukemia, lymphocytic lymphoma, kidney or ureter cancer, renal cell carcinoma, renal pelvic carcinoma, central nervous system tumor, primary central nervous system lymphoma, spinal cord tumor, brainstem glioma and pituitary adenoma.

In one embodiment, the composition of the present disclosure may further comprise an immunogenic cell death inducing agent. The immunogenic cell death inducing agent may be any one or more selected from the group consisting of anthracycline-based anticancer agents, taxane-based anticancer agents, anti-EGFR antibodies, BK channel agonists, bortezomib, cardiac glycoside, cyclophosmide anticancer agents, GADD34/PP1 inhibitors, LV-tSMAC, Measles virus, bleomycin, mitoxantrone, or oxaliplatin. The anthracycline-based anticancer agent may be daunorubicin, doxorubicin, epirubicin, idarubicin, pixantrone, sabarubicin, or valrubicin, and the taxane-based anticancer agent may be paclitaxel or docetaxel.

The pharmaceutical composition of the present disclosure may be used as a single therapy, but may also be used in combination with other conventional biological therapy, chemotherapy or radiation therapy, and may treat more effectively cancer in the case of such combined therapy.

The pharmaceutical composition for preventing or treating cancer of the present disclosure is administered together with a chemical anticancer drug (anticancer agent) and the like to increase a cancer therapy effect of conventional anticancer agents through the death effect of cancer cells. Combined administration may be performed simultaneously or sequentially with the anticancer agent. Examples of the anticancer agent include DNA alkylating agents such as mechloethamine, chlorambucil, phenylalanine, mustard, cyclophosphamide, ifosfamide, carmustine (BCNU), lomustine (CCNU), streptozotocin, busulfan, thiotepa, cisplatin, and carboplatin: anti-cancer antibiotics such as dactinomycin (actinomycin D), plicamycin, and mitomycin C; plant alkaloids such as vincristine, vinblastine, etoposide, teniposide, topotecan, and iridotecan, and the like, but are not limited thereto.

As used in the present disclosure, the term “prevention” means all actions that inhibit or delay the occurrence, spread, and recurrence of cancer by administration of the pharmaceutical composition according to the present disclosure.

As used in the present disclosure, the term “treatment” refers to all actions that improve or beneficially change the death of cancer cells or the symptoms of cancer by administration of the composition of the present disclosure. Those skilled in the art to which the present disclosure pertains will be able to determine the degree of improvement, enhancement and treatment by knowing the exact criteria of disease for which the composition of the present disclosure is effective by referring to data presented by the Korean Academy of Medical Sciences, etc.

As used in the present disclosure, the term “therapeutically effective amount” used in combination with the active ingredient means an amount of pharmaceutically acceptable salt of the composition effective for preventing or treating a target disease, and the therapeutically effective amount of the composition of the present disclosure may vary depending on many factors, such as a method of administration, a target site, the condition of a patient, and the like. Accordingly, when used in the human body, a dose should be determined as an appropriate amount in consideration of both safety and efficiency. It is also possible to estimate the amount used in humans from the effective amount determined through animal experiments. These matters to be considered when determining the effective amount are described in, for example, Hardman and Limbird, eds., Goodman and Gilman's The Pharmacological Basis of Therapeutics, 10th ed. (2001), Pergamon Press; and E. W. Martin ed., Remington's Pharmaceutical Sciences, 18th ed. (1990), Mack Publishing Co.

The pharmaceutical composition of the present disclosure is administered in a pharmaceutically effective amount. As used in the present disclosure, the term “pharmaceutically effective amount” refers to an amount enough to treat the disease at a reasonable benefit/risk ratio applicable to medical treatment and enough not to cause side effects. The effective dose level may be determined according to factors including the health condition of a patient, the type and severity of cancer, the activity of a drug, the sensitivity to a drug, a method of administration, a time of administration, a route of administration, an excretion rate, duration of treatment, and drugs used in combination or simultaneously, and other factors well-known in the medical field. The composition of the present disclosure may be administered as an individual therapeutic agent or in combination with other therapeutic agents, and may be administered sequentially or simultaneously with conventional therapeutic agents, and may be administered singly or multiply. It is important to administer an amount capable of obtaining a maximum effect with a minimal amount without side effects by considering all the factors, which may be easily determined by those skilled in the art.

The pharmaceutical composition of the present disclosure may further comprise a pharmaceutically acceptable additive. At this time, the pharmaceutically acceptable additive may be used with starch, gelatinized starch, microcrystalline cellulose, lactose, povidone, colloidal silicon dioxide, calcium hydrogen phosphate, lactose, mannitol, syrup, arabic gum, pregelatinized starch, corn starch, powdered cellulose, hydroxypropyl cellulose, Opadry, sodium starch glycolate, lead carnauba, synthetic aluminum silicate, stearic acid, magnesium stearate, aluminum stearate, calcium stearate, white sugar, dextrose, sorbitol, talc and the like. The pharmaceutically acceptable additive according to the present disclosure is preferably included in an amount of 0.1 parts by weight to 90 parts by weight based on the composition, but is not limited thereto.

The composition of the present disclosure may include a carrier, a diluent, an excipient, or a combination of two or more thereof, which are commonly used in biological agents. The pharmaceutically acceptable carrier is not particularly limited as long as the carrier is suitable for in vivo delivery of the composition, and may be used by combining, for example, compounds described in Merck Index, 13th ed., Merck & Co. Inc., saline, sterile water, a Ringer's solution, buffered saline, a dextrose solution, a maltodextrin solution, glycerol, ethanol, and one or more of these ingredients, and if necessary, other conventional additives such as an antioxidant, a buffer, and a bacteriostat may be added. In addition, the pharmaceutical composition may be prepared in injectable formulations such as an aqueous solution, a suspension, and an emulsion, pills, capsules, granules, or tablets by further adding a diluent, a dispersant, a surfactant, a binder, and a lubricant. Furthermore, the pharmaceutical composition may be prepared preferably according to each disease or ingredient using a suitable method in the art or a method disclosed in Remington's Pharmaceutical Science (Mack Publishing Company, Easton PA, 18th, 1990).

The composition of the present disclosure may be administered parenterally (e.g., applied with injectable formulations intravenously, subcutaneously, intraperitoneally or topically) or orally according to a desired method, and the range of the dose may vary depending on the body weight, age, sex, and health condition of a patient, a diet, an administration time, an administration method, an excretion rate, the severity of a disease, and the like. A daily dose of the composition according to the present disclosure is 0.0001 to 10 mg/ml, preferably 0.0001 to 5 mg/ml, and more preferably administered once to several times a day.

Liquid formulations for oral administration of the composition of the present disclosure correspond to suspensions, internal solutions, emulsions, syrups, etc., and may include various excipients, such as wetting agents, sweeteners, fragrances, preservatives, and the like, in addition to water and liquid paraffin, which are commonly used simple diluents. Formulations for parenteral administration include sterilized aqueous solutions, non-aqueous solvents, suspensions, emulsions, lyophilized agents, suppositories, and the like.

In an aspect, the present disclosure relates to a method for preparing a human antibody Fc domain variant comprising a) incubating a host cell including a vector including a nucleic acid molecule encoding the human antibody Fc domain variant of the present disclosure; and b) recovering a polypeptide expressed by the host cell.

In one aspect, the present disclosure relates to a method for preparing an antibody with a reduced effector function comprising a) incubating a host cell including a vector including a nucleic acid molecule encoding the antibody of the present disclosure or immunologically active fragment thereof; and b) purifying the antibody expressed by the host cell.

In one embodiment, the purification of the antibody may include filtration, HPLC, anion exchange or cation exchange, high performance liquid chromatography (HPLC), affinity chromatography, or a combination thereof, preferably affinity chromatography using Protein A.

In one aspect, the present disclosure relates to a use of the Fc domain variant of the present disclosure, the antibody including the Fc domain variant or the immunologically active fragment thereof for preparation of an antibody therapeutic agent.

In one aspect, the present disclosure relates to a use for preventing or treating of cancer of the Fc domain variant of the present disclosure, the antibody including the Fc domain variant or the immunologically active fragment thereof, or the antibody therapeutic agent in which the antibody or immunologically active fragment thereof is conjugated to one or more therapeutic agents.

In one aspect, the present disclosure relates to a method of treating cancer comprising administering the Fc domain variant of the present disclosure, the antibody including the Fc domain variant or the immunologically active fragment thereof, or the antibody therapeutic agent in which the antibody or immunologically active fragment thereof is conjugated to one or more therapeutic agents in a pharmaceutically effective amount, to a subject with cancer.

### Modes of the Invention

Hereinafter, the present disclosure will be described in more detail with reference to the following Examples. However, the following Examples are only intended to embody the contents of the present disclosure, and the present disclosure is not limited thereto.

### Example 1. Construction of Mammalian Cell Display System for Screening Glycosylated Fc Variants

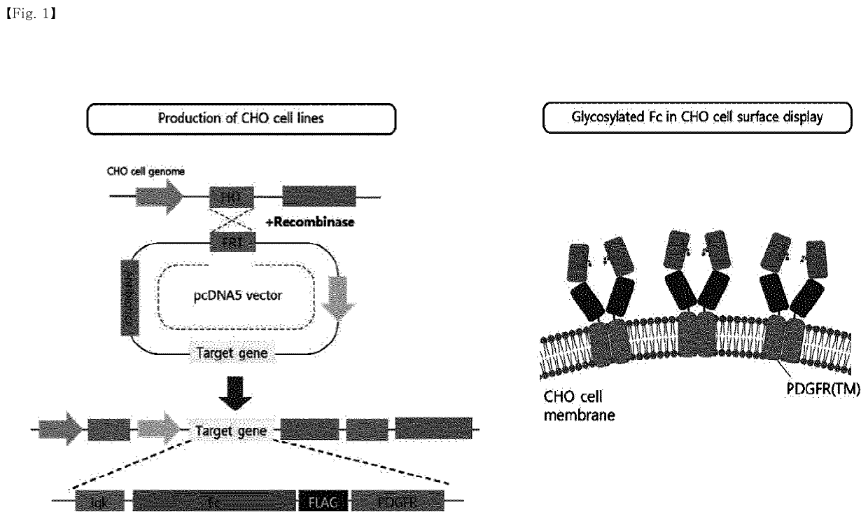

An Fc library was constructed to discover novel glycosylated Fc variants capable of removing an immune action mechanism. Specifically, to display and stably screen the glycosylated Fc variants on the surface of a glycosylated Fc mammalian cell, a FLP-FRT gene recombination system, which was used to construct stable cell lines, was used, and a platelet-derived growth factor receptor (PDGFR) transmembrane domain was fused to the C-terminus of Fc to be displayed on the cell membrane. An Fc-PDGFR gene was cloned and prepared into pcDNA5/FRT plasmid (Invitrogen, V601020) into which a FRT site was inserted. The corresponding plasmid was co-transfected into CHO cells (Invitrogen, R75807) into which the FRT site was inserted, together with a pOG44 plasmid (Invitrogen, V600520) expressing FLP, an enzyme causing genetic recombination, to induce gene recombination, and thus a CHO cell line stably expressing glycosylated Fc was prepared by integrating Fc-PDGFR DNA into chromosomal DNA of CHO cells (FIG. 1). At this time, in order to sort only CHO cells in which gene integration occurred after transfection, a hygromycin-B resistance gene was integrated together, and a CHO cell line stably expressing glycosylated Fc was sorted and prepared by treatment with 500 μg/ml of hygromycin-B (Invitrogen, 10687010).

### Example 2. Expression and Purification of FcγRs

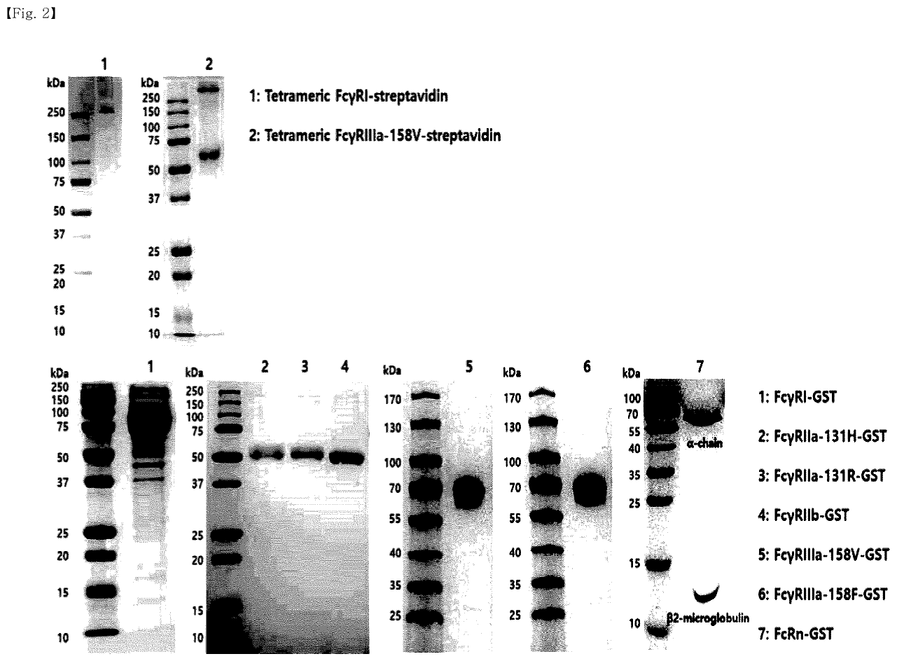

In order to perform FACS screening of the CHO cell display system for the glycosylated Fc established in Example 1 and the Fc library stable expression cell line (stable cell line), FcγRI and FcγRIIIa were produced to remove binding affinity for FcγRI having the highest binding affinity among human FcγRs and FcγRIIIa involved in ADCC in the immune action mechanism. Specifically, tetrameric FcγRI-streptavidin-His and tetrameric FcγRIIIa-158V-streptavidin-His were prepared by fusing streptavidin to the C-terminus of each receptor to enable efficient FACS screening by enhancing the visible binding affinity for Fc. In addition, FcγRI-GST, FcγRIIa-131H-GST, FcγRIIa-131R-GST, FcγRIIb-GST, FcγRIIIa-158V-GST, and FcγRIIIa-158F-GST were prepared and produced to analyze the binding affinity of the discovered glycosylated Fc variants for FcγRs by ELISA. In addition, FcRn-GST necessary for analyzing the FcRn binding affinity involved in the in vivo half-life of the antibody was prepared and produced. Each protein produced above was cloned into an animal cell expression vector, transfected into Expi293F cells using PEI, and incubated for 7 days under conditions of 37° C., 125 rpm, and 8% CO₂. After incubating, the supernatant was recovered, equilibrated with PBS, purified by Ni-NTA (Anti-His) or anti-GST affinity chromatography, and then confirmed by SDS-PAGE gel. As a result, it was confirmed that tetrameric FcRI-streptavidin, tetrameric FcγRIIIa-158V-streptavidin, FcγRI-GST, FcγRIIa-131H-GST, FcγRIIa-131R-GST, FcγRIIb-GST, FcγRIIIa-158V-GST, FcγRIIIa-158F-GST, and FcRn-GST were purified with high purity (FIG. 2).

### Example 3. Labeling and Validation of FcγRs

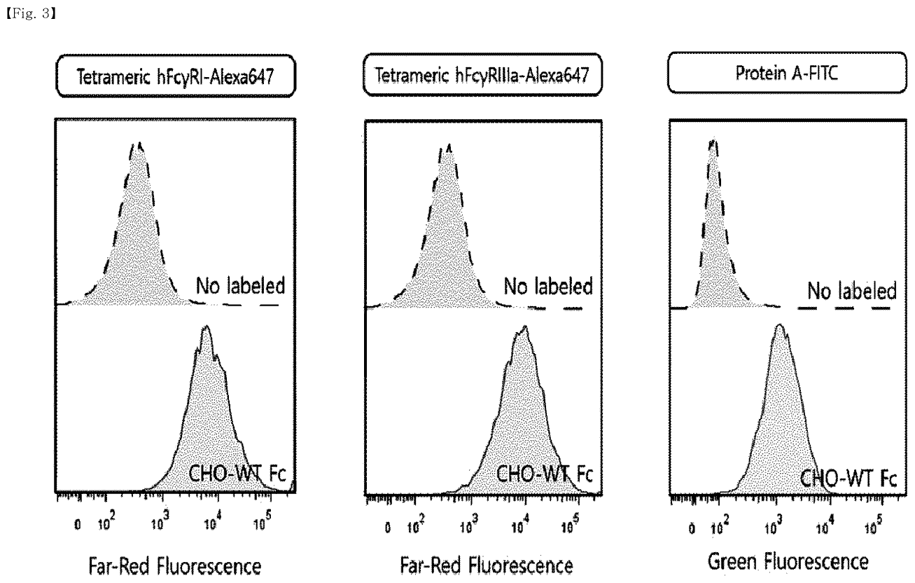

Among the produced proteins, tetrameric FcγRI-streptavidin and tetrameric FcγRIIIa-streptavidin prepared for FACS screening were labeled with Alexa647 (Invitrogen, A20173) fluorescent dye to enable sorting to isolate Fc variants binding to the dye. In addition, in order to confirm the expression and the expression levels of the displayed Fc variants, FITC (Invitrogen, F6434) was conjugated to Protein A (Amicogen, 1070020), which did not overlap with the binding sites of FcγRs. The conjugation of fluorescent dyes Alexa647 and FITC was performed according to the manual provided by each manufacturer. Through this, the fluorescently labeled tetrameric FcγRI-streptavidin-Alexa647, tetrameric FcγRIIIa-streptavidin-Alexa647, and Protein A-FITC were induced to bind to wild-type Fc displayed on CHO cells, and then the activity thereof was confirmed. As a result, it was confirmed that Protein A-FITC had normal activity through a fluorescence signal by the Protein A-FITC binding, and that the wild-type Fc displayed on the CHO cells was stably expressed (FIG. 3). In addition, it was shown that tetrameric FcγRI-streptavidin-Alexa647 and tetrameric FcγRIIIa-streptavidin-Alexa647 also bound normally to wild-type Fc (FIG. 3), and it was validated that Alexa647 fluorescently-labeled FcγRI and FcγRIIIa also had excellent activity, and that Fcs displayed on the CHO cells were normally expressed and performed their respective functions.

### Example 4. Construction of Glycosylated Fc Variant Library with Removed FcγRs Binding Affinity Using CHO Cell Display

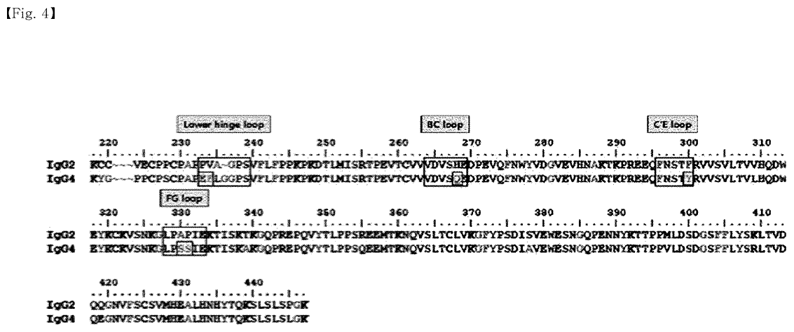

Glycosylated Fc was engineered using an established CHO cell Fc display system and a library was constructed to screen glycosylated Fc variants with removed binding affinity to FcγRs. The library was constructed by introducing mutations at six sites, excluding the existing mutation sites based on IgG2 and IgG4 sequences to discover novel mutations capable of completely removing FcγRs binding, assuming that the amino acids in IgG2 with the lowest binding affinity for FcγRs in four regions (lower hinge, B/C loop, C′/E loop, and F/G loop) of Fc, which has a critical effect on the binding to FcγRs, had a critical effect on the non-binding to FcγRs (FIG. 4). The library genes were co-transfected with an FLP expression plasmid in the same manner as the method for preparing the Fc stable expression CHO cell line established in Example 2 above, and then subjected to a screening process using a hygromycin-B medium to construct a glycosylated Fc variant library stable expression CHO cell line.

### Example 5. Sorting of Fc Variants with Removed FcγRs Binding Affinity Using CHO Cell Display

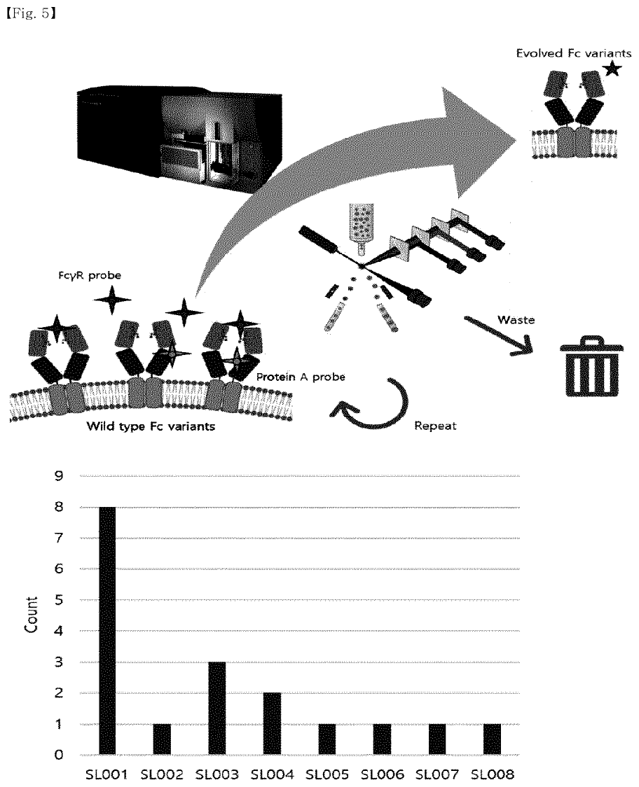

To sort glycosylated Fc with removed FcγRIIIa binding affinity using the established CHO cell Fc display system and the Fc library stable expressing CHO cell line, binding of FcγRIIIa-Alexa647 and Protein A-FITC was induced in the library stable expressing CHO cell line, and then the expression level by Protein A-FITC and the removed FcγRIIIa binding were simultaneously monitored to perform FACS sorting of 1R. Thereafter, FACS sorting of 2R was performed using the same method to sort the glycosylated Fc with removed FcγRI binding affinity from the library that had undergone 1R, and FACS sorting of 3R using FcγRIIIa-Alexa647 and Protein A-FITC, and FACS sorting of 4R using FcγRI-Alexa647 and Protein A-FITC were performed using the same method. As a result, the population to be expected to exhibit the removed FcγRI binding affinity and FcγRIIIa binding affinity was sorted, and the sorted glycosylated Fc variant-expressing CHO cells were recovered through genomic DNA prep, and then nucleotide sequences were confirmed to finally sort the engineered glycosylated Fc variants (FIG. 5 and Table 1).

TABLE 1

| Fc variants | Mutations |
|---|---|
| IgG4 wild-type | — |
| S228P | S228P |
| SL001 | E233C |
| SL002 | E233P/P291S |
| SL003 | E233G |
| SL004 | E233G/L309P |
| SL005 | F234G |
| SL006 | F234G/G402D |
| SL007 | F234T |
| SL008 | F234R |

### Example 6. Expression and Purification of Glycosylated Pembrolizumab Fc Variants Introduced with Sorted Fc Variants

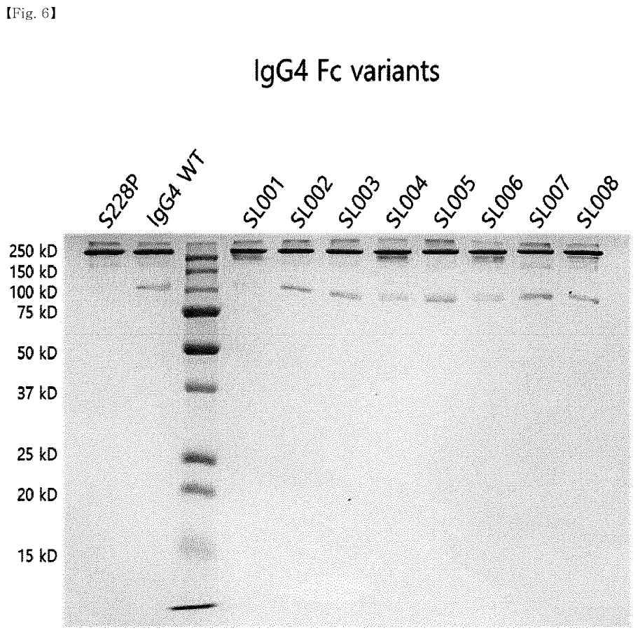

The sorted glycosylated Fc variants were cloned into a pembrolizumab heavy chain gene, which did not have a Fab-arm exchange variant as a model antibody to produce an expression vector. Thereafter, heavy chain genes and light chain genes of the variants were first mixed in 3 ml of a Freestyle 293 expression culture medium (Gibco, 12338-018) at a 1:1 ratio, and mixed at a ratio of PEI:variant genes=4:1, left at room temperature for 20 minutes, and then transfected into Expi293F cells subcultured at the previous day with a density of 2×10⁶ cells/ml. Thereafter, the transfected cells were incubated for 7 days under conditions of 37° C., 125 rpm, and 8% CO₂ in a CO₂ shaking incubator, and then centrifuged to collect only a supernatant. The supernatant was equilibrated with 25×PBS and then filtered and prepared through a 0.2 μm syringe filter. Thereafter, Protein A resin was added to the culture medium containing pembrolizumab Fc variants, stirred at 4° C. for 16 hours, and then spun down to recover a resin, washed with 2 ml of PBS, and eluted with 300 μl of a 100 mM glycine (pH 2.7) buffer. Thereafter, the solution was neutralized using 100 ul of 1 M Tris-HCl (pH 8.0) and the buffer was replaced using Amicon Ultra-4 centrifugal filter units 30K (Merck Millipore, UFC800324). Through SDS-PAGE gel analysis, it was confirmed that the glycosylated antibody pembrolizumab Fc variants were purified with high purity (FIG. 6).

### Example 7. Confirmation of Binding Affinity for FcγRs of Glycosylated Pembrolizumab Fc Variants

To confirm the binding affinity for FcγRI and FcγRIIIa of the glycosylated pembrolizumab Fc variants purified in Example 6 above, ELISA analysis was performed. Specifically, 50 μl of FcγRs-GSTs (FcγRIIb-GST and FcγRIIIa-158V-GST) diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. The plate was washed four times with 180 μl of 0.05% PBST, and then 50 μl of glycosylated pembrolizumab Fc variants serially diluted with 1% skim milk were dispensed into each well and reacted at room temperature for 1 hour. After washing, antibody reaction was performed for 1 hour at room temperature using 50 μl of HRP-Protein L (GenScript, M00098) and then washed again. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

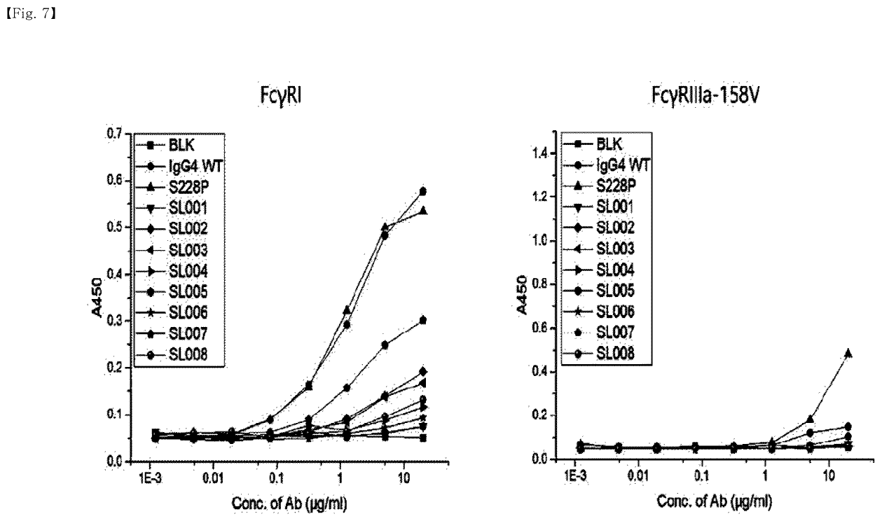

As a result, the glycosylated pembrolizumab Fc variants discovered through Examples above were approved by the US FDA as various cancer therapeutic agents and were shown to have significantly lower binding affinity for FcγRI and FcγRIIIa-158V than pembrolizumab with a S228P mutation, and in particular, a SL001 variant (E233C) and a SL005 variant (F234G) were shown to have removed binding affinity for FcγRI (FIG. 7).

### Example 8. Recombination of Sorted Pembrolizumab Fc Variants and Fab-Arm Exchange-Prevention Variants

Wild-type human IgG4 had serine, an amino acid at position 228 of a CH2 domain, unlike human IgG1, and an intrachain disulfide bond was formed through a flexible core hinge to produce a half antibody with a non-covalent linkage. IgG4, which existed in the half-antibody form, exhibited a Fab-arm exchange phenomenon in which two half-antibody forms of IgG4 targeting different antigens were combined. To prevent this, when serine at position 288 of IgG4 was replaced with proline, which was an amino acid at position 228 of human IgG1, a hinge region was stabilized and Fab-arm exchange did not occur, so that the IgG4 was generally applied to IgG4 clinical antibodies and then developed. Accordingly, the S228P mutation was introduced into the glycosylated pembrolizumab Fc variants discovered in Examples above, and recombined with the glycosylated pembrolizumab Fc variants SPEC, SPFG, and SPECFG to confirm a combination effect of SL001 (E233C) and SL005 (F234G) (Table 2).

TABLE 2

| Fc variants | Mutations |
|---|---|
| IgG4 wild-type | |
| S228P | S228P |
| SPLE | S228P/L235E |
| SPEC | S228P/E233C |
| SPFG | S228P/F234G |
| SPECFG | S228P/E233C/F234G |

### Example 9. Expression and Purification of Recombinant Glycosylated Pembrolizumab Fc Variants for Fab-Arm Exchange Prevention

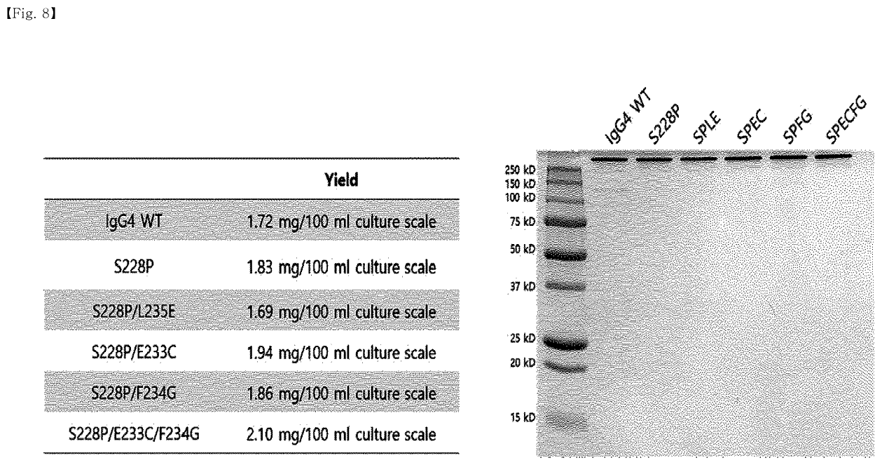

The recombinant glycosylated Fc variants in Table 2 above were cloned into a heavy chain gene of a model antibody, pembrolizumab, to prepare an expression vector. Thereafter, heavy chain genes and light chain genes of the variants were first mixed in 10 ml of a Freestyle 293 expression culture medium (Gibco, 12338-018) at a 1:1 ratio, and mixed at a ratio of PEI:variant gene=4:1, left at room temperature for 20 minutes, and then transfected into Expi293F cells subcultured at the previous day with a density of 2×10⁶ cells/ml. Thereafter, the transfected cells were incubated for 7 days under conditions of 37° C., 125 rpm, and 8% CO₂ in a CO₂ shaking incubator, and then centrifuged to collect only a supernatant. The supernatant was equilibrated with 25×PBS and then filtered and prepared through a 0.2 μm syringe filter. Thereafter, Protein A resin was added to the culture medium containing the recombinant pembrolizumab Fc variants, stirred at 4° C. for 16 hours, and then spun down to recover a resin, washed with 10 ml of PBS, and eluted with 3 ml of a 100 mM glycine (pH 2.7) buffer. Thereafter, the solution was neutralized using 1 ml of 1 M Tris-HCl (pH 8.0) and the buffer was replaced using Amicon Ultra-4 centrifugal filter units 30K (Merck Millipore, UFC800324). Thereafter, through SDS-PAGE gel analysis, it was confirmed that the glycosylated antibody pembrolizumab Fc variants were purified with high purity, and that the expression levels thereof were superior to those of wild-type IgG4 and the model antibody pembrolizumab (FIG. 8).

### Example 10. Confirmation of Binding Affinity for FcγRs of Recombinant Glycosylated Pembrolizumab Fc Variants for Fab-Arm Exchange Prevention

To confirm the binding affinity for FcγRs of the recombinant glycosylated pembrolizumab Fc variants purified in Example 9 above, ELISA analysis was performed. Specifically, 50 μl of FcγRs-GSTs (FcγRI-GST, FcγRIIa-131H-GST, FcγRIIa-131R-GST, FcγRIIb-GST, FcγRIIIa-158V-GST and FcγRIIIa-158F-GST) diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. The plate was washed four times with 180 μl of 0.05% PBST, and then 50 μl of glycosylated pembrolizumab Fc variants serially diluted with 1% skim milk were dispensed into each well and reacted at room temperature for 1 hour. After washing, antibody reaction was performed for 1 hour at room temperature using 50 μl of HRP-Protein L (GenScript, M00098) and then washed again. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

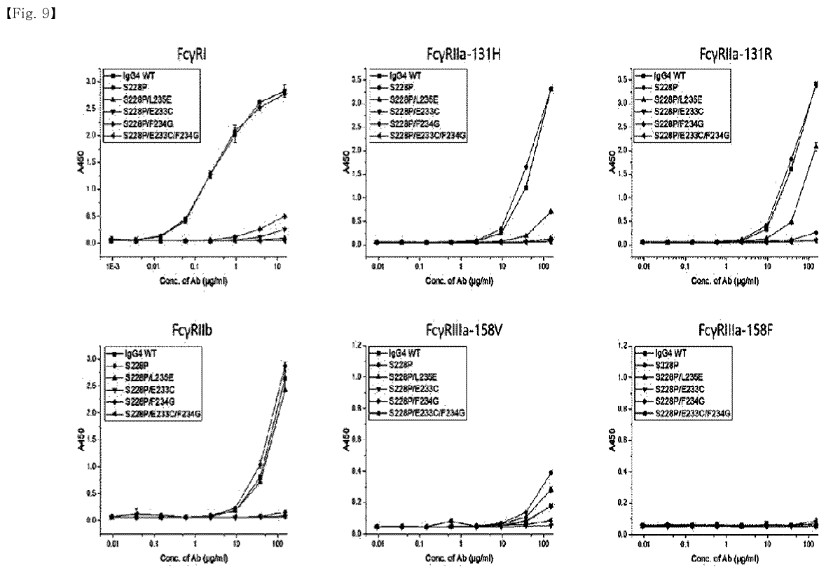

As a result, the recombinant glycosylated pembrolizumab Fc variants of the present disclosure were approved by the US FDA as wild-type IgG4 and various cancer therapeutic agents and shown to have significantly lower FcγRs binding affinity than pembrolizumab having the S228P mutation. In particular, the conventional S228P variant (pembrolizumab) and the S228P/L235E variant were shown to retain binding affinity for FcγRs, but the SPECFG variant (S228P/E233C/F234G) was shown to have removed binding affinity to all FcγRs (FIG. 9).

### Example 11. Confirmation of C1q Binding Affinity of Recombinant Glycosylated Pembrolizumab Fc Variants for Fab-Arm Exchange Prevention

To confirm C1q binding affinity of the recombinant glycosylated pembrolizumab Fc variants purified in Example 9 above, ELISA analysis was performed. Specifically, 50 μl of the glycosylated pembrolizumab Fc variants diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. The plate was washed four times with 180 μl of 0.05% PBST, and then 50 μl of C1q (Quidel, A400) protein serially diluted with 1% skim milk were dispensed into each well and reacted at room temperature for 1 hour. After washing, antibody reaction was performed for 1 hour at room temperature using 50 μl of anti-C1q-HRP (Invitrogen, PA1-84324) and then washed. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

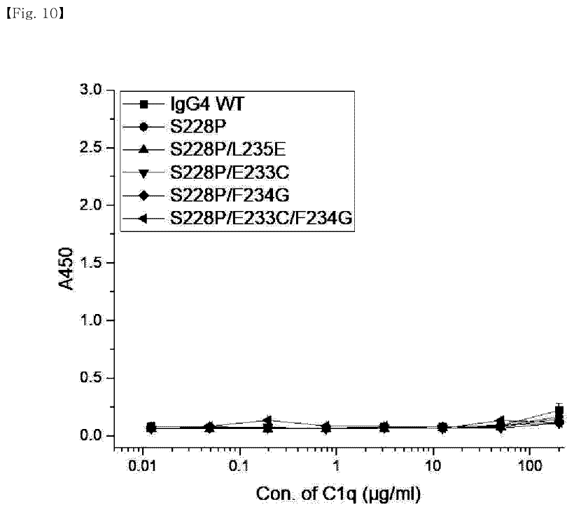

As a result, it was shown that the recombinant glycosylated pembrolizumab Fc variants of the present disclosure had no binding affinity for C1q (FIG. 10).

### Example 12. Confirmation of FcRn Binding Affinity of Recombinant Glycosylated Pembrolizumab Fc Variants for Fab-Arm Exchange Prevention

To confirm binding affinity for FcRn involved in of the in vivo half-life of the recombinant glycosylated pembrolizumab Fc variants purified in Example 9 above, ELISA analysis according to pH was performed. Specifically, 50 μl of the glycosylated pembrolizumab Fc variants diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. After washed 4 times with 180 μl of 0.05% PBST (pH 6.0/pH 7.4), 50 μl of FcRn-GST serially diluted with 1% skim milk (pH 6.0/pH 7.4) was dispensed into each well and reacted at room temperature for 1 hour. After washing, antibody reaction was performed for 1 hour at room temperature using 50 μl of anti-GST-HRP Conjugate (GE Healthcare, RPN1236V) and then washed again. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

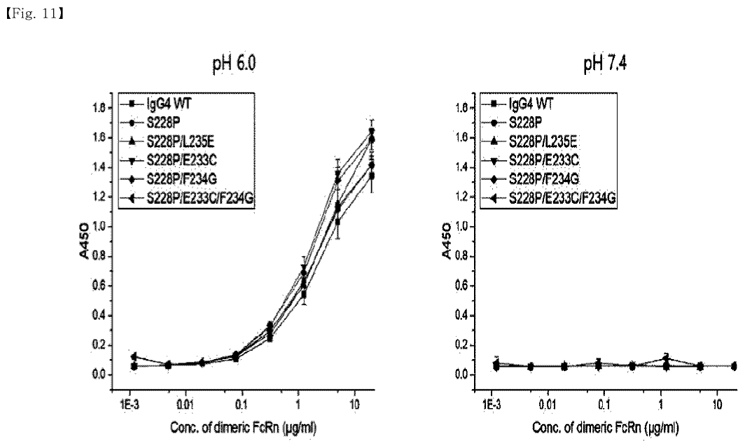

As a result, it was shown that the recombinant glycosylated pembrolizumab Fc variants of the present disclosure maintained the FcRn binding properties of wild-type IgG4 (FIG. 11).

### Example 13. Thermal Stability Analysis of Recombinant Glycosylated Pembrolizumab Fc Variants for Fab-Arm Exchange Prevention

To confirm thermal stability of the recombinant glycosylated pembrolizumab Fc variants purified in Example 9 above according to a temperature, differential scanning fluorimetry (DSF) analysis was performed. Specifically, 45 μl of the glycosylated pembrolizumab Fc variants diluted to 5 μM in 1×PBS and 5 μl of SYPRO Orange (Invitrogen, S6651) dye diluted to 200× were mixed and dispensed onto a PCR plate (Thermo Scientific, AB0900W), respectively. 1×PBS was also prepared as a control in the same manner (all samples were performed in a triplicate). An optically clear sealing film (Thermo Scientific, AB1170) was attached to the plate containing the samples, and the fluorescence intensity was measured at a temperature increase of 0.03° C. per second from 25° C. to 99.9° C. using a QuantStudio 3 Real-Time PCR System (Applied Biosystems, A28567). The fluorescence values according to each temperature were fitted to a Boltzmann model using OriginPro software, and then the midpoint of a sigmoidal transition curve was calculated.

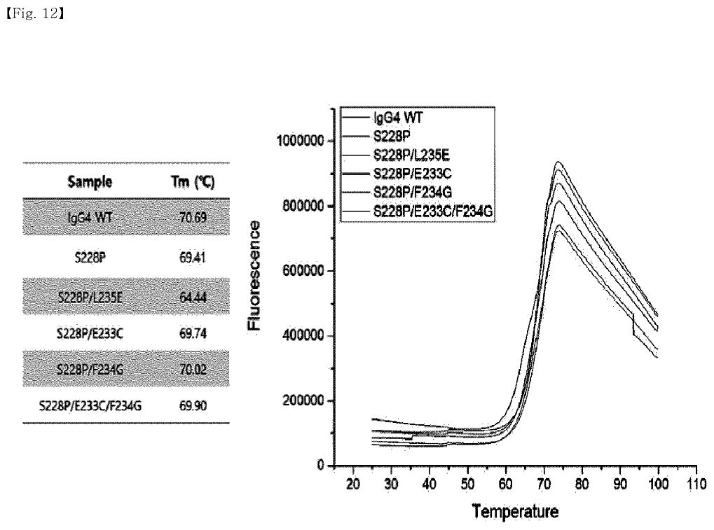

As a result, the recombinant glycosylated pembrolizumab Fc variants of the present disclosure had a degradation temperature similar to that of wild-type IgG4, and the conventional S228P/L235E variant showed a decreased degradation temperature (FIG. 12).

### Example 14. Preparation of Combined Variants of Glycosylated Pembrolizumab Fc Variants with Removed Binding Affinity for FcγRI

To confirm a combination effect of the SL001 variant (E233C) and the SL005 variant (F234G), which were sorted as glycosylated pembrolizumab Fc variants with removed binding affinity for FcγRI in Example 7 above, a combined pembrolizumab Fc variant ECFG (E233C/F234G) was prepared (Table 3).

TABLE 3

| Fc variants | Mutations |
|---|---|
| IgG4-ECFG | E233C/F234G |

### Example 15. Expression and Purification of Combined Glycosylated Pembrolizumab Fc Variants

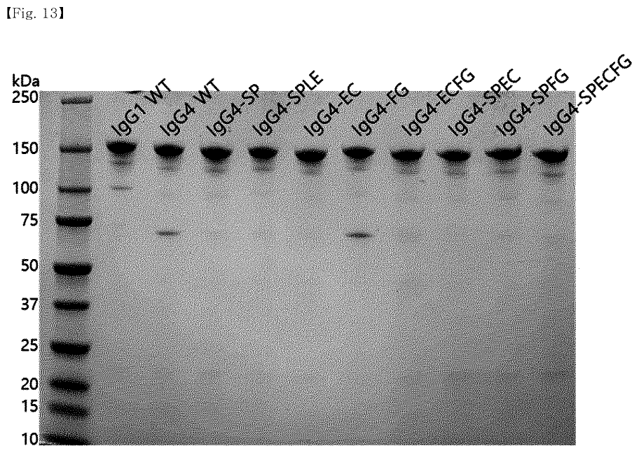

The combined glycosylated Fc variant ECFG of Table 3 above and the recombinant glycosylated Fc variants SPLE, SPEC, SPFG, and SPECFG of Table 2 were cloned into the heavy chain gene of a model antibody, pembrolizumab to prepare an expression vector. Thereafter, heavy chain genes and light chain genes of the variants were first mixed in 10 ml of a Freestyle 293 expression culture medium (Gibco, 12338-018) at a 1:1 ratio, and mixed at a ratio of PEI:variant gene=4:1, left at room temperature for 20 minutes, and then transfected into Expi293F cells subcultured at the previous day with a density of 2×10⁶ cells/ml. Thereafter, the transfected cells were incubated for 7 days under conditions of 37° C., 125 rpm, and 8% CO₂ in a CO₂ shaking incubator, and then centrifuged to collect only a supernatant. The supernatant was equilibrated with 25×PBS and then filtered and prepared through a 0.2 μm syringe filter. Thereafter, Protein A resin was added to the culture medium containing the pembrolizumab Fc variants, stirred at 4° C. for 16 hours, and then spun down to recover a resin, washed with 10 ml of PBS, and eluted with 3 ml of a 100 mM glycine (pH 2.7) buffer. Thereafter, the solution was neutralized using 1 ml of 1 M Tris-HCl (pH 8.0) and the buffer was replaced using Amicon Ultra-4 centrifugal filter units 30K (Merck Millipore, UFC800324). Thereafter, it was confirmed through SDS-PAGE gel analysis that the glycosylated antibody pembrolizumab Fc variants of the present disclosure were purified with high purity, and it was confirmed that the wild-type IgG4 antibody formed a half-antibody of 75 kDa, but model antibodies pembrolizumab (IgG4-SP), IgG4-SPLE (S228P/L235E), IgG4-SPEC (S228P/E233C), IgG4-SPFG (S228P/F234G), and IgG4-SPECFG (S228P/E233C/F234G) into which the Fab-arm exchange-preventing variant S228P was introduced did not form half-antibodies. In addition, it was confirmed that IgG4-EC (SL001 and E233C in Table 1) and IgG4-ECFG (E233C/F234G) did not show a Fab-arm exchange phenomenon and did not form half-antibodies even though the Fab-arm exchange-preventing variant (S228P) was not introduced (FIG. 13).

### Example 16. Confirmation of Binding Affinity for Human FcγRs of Glycosylated Pembrolizumab Fc Variants

To confirm the binding affinity for FcγRs of the combined glycosylated pembrolizumab Fc variants and the recombinant glycosylated Fc variants purified in Example 15 above, ELISA analysis was performed. Specifically, 50 μl of FcγRs-GSTs (FcγRI-GST, FcγRIIa-131H-GST, FcγRIIa-131R-GST, FcγRIIb-GST, FcγRIIIa-158V-GST and FcγRIIIa-158F-GST) diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. The plate was washed four times with 180 μl of 0.05% PBST, and then 50 μl of the glycosylated pembrolizumab Fc variants serially diluted with 1% skim milk were dispensed into each well and reacted at room temperature for 1 hour. After washing, antibody reaction was performed for 1 hour at room temperature using 50 μl of HRP-Protein L (GenScript, M00098) and then washed again. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

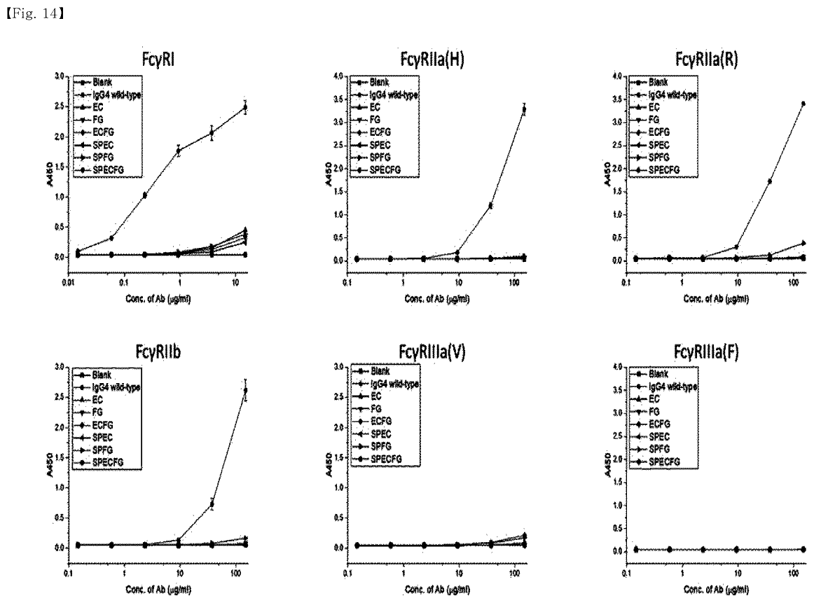

As a result, the glycosylated pembrolizumab Fc variants of the present disclosure were found to have significantly lower FcγRs binding affinity than the wild-type IgG4 antibody, and in particular, the ECFG variant (E233C/F234G) and the SPECFG variant (S228P/E233C/F234G) were found to have removed binding affinity for all FcγRs (FIG. 14).

### Example 17. Confirmation of Binding Affinity for Human C1q of Glycosylated Pembrolizumab Fc Variants

To confirm C1q binding affinity of the recombinant glycosylated pembrolizumab Fc variants purified in Example 15 above, ELISA analysis was performed. Specifically, 50 μl of the glycosylated pembrolizumab Fc variants diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. The plate was washed four times with 180 μl of 0.05% PBST, and then 50 μl of C1q (Quidel, A400) protein serially diluted with 1% skim milk were dispensed into each well and reacted at room temperature for 1 hour. After washing, antibody reaction was performed for 1 hour at room temperature using 50 μl of anti-C1q-HRP (Invitrogen, PA1-84324) and then washed. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

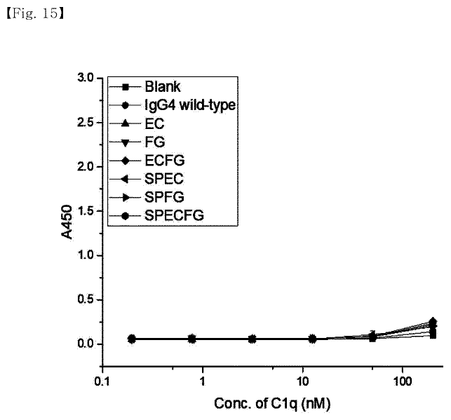

As a result, it was shown that the glycosylated pembrolizumab Fc variants EC, FG, ECFG, SPEC, SPFG, and SPECFG of the present disclosure had no binding affinity for C1q (FIG. 15).

### Example 18. Confirmation of Binding Affinity for Human FcRn of Glycosylated Pembrolizumab Fc Variants

To confirm binding affinity for FcRn involved in of the in vivo half-life of the glycosylated pembrolizumab Fc variants purified in Example 15 above, ELISA analysis according to pH was performed. Specifically, 50 μl of the glycosylated pembrolizumab Fc variants diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. After washed 4 times with 180 μl of 0.05% PBST (pH 6.0/pH 7.4), 50 μl of FcRn-GST serially diluted with 1% skim milk (pH 6.0/pH 7.4) was dispensed into each well and reacted at room temperature for 1 hour. After washing, antibody reaction was performed for 1 hour at room temperature using 50 μl of anti-GST-HRP Conjugate (GE Healthcare, RPN1236V) and then washed again. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

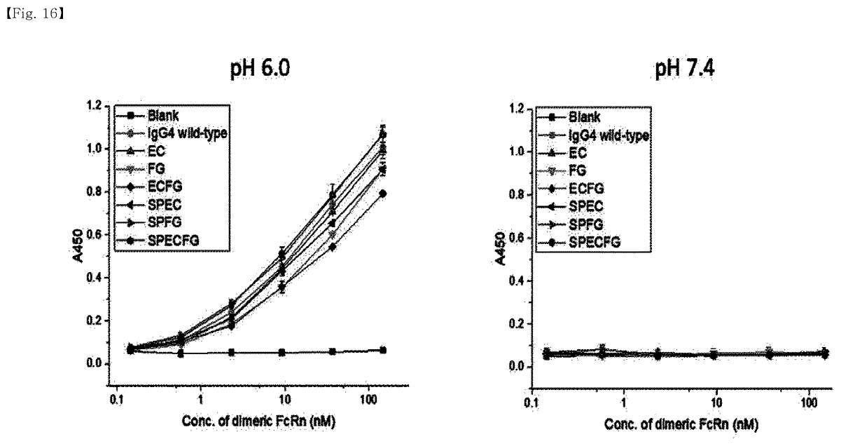

As a result, it was shown that the glycosylated pembrolizumab Fc variants EC, FG, ECFG, SPEC, SPFG, and SPECFG of the present disclosure maintained the FcRn binding properties of wild-type IgG4 (FIG. 16).

### Example 19. Thermal Stability Analysis of Glycosylated Pembrolizumab Fc Variants

To confirm thermal stability of the glycosylated pembrolizumab Fc variants purified in Example 15 above according to a temperature, DSF analysis was performed. Specifically, 45 μl of the glycosylated pembrolizumab Fc variants diluted to 5 μM in 1×PBS and 5 μl of SYPRO Orange (Invitrogen, S6651) dye diluted to 200× were mixed and dispensed onto a PCR plate (Thermo Scientific, AB0900W), respectively. 1×PBS was also prepared as a control in the same manner (all samples were performed in a triplicate). An optically clear sealing film (Thermo Scientific, AB1170) was attached to the plate containing the samples, and the fluorescence intensity was measured at a temperature increase of 0.03° C. per second from 25° C. to 99.9° C. using a QuantStudio 3 Real-Time PCR System (Applied Biosystems, A28567). The fluorescence values according to each temperature were fitted to a Boltzmann model using OriginPro software, and then the midpoint of a sigmoidal transition curve was calculated.

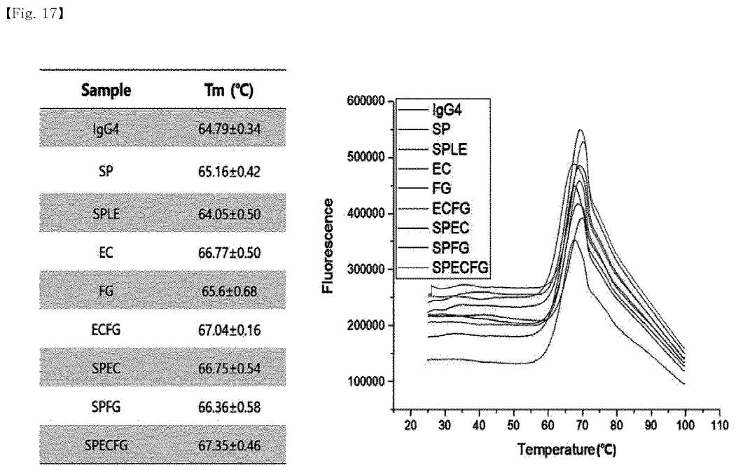

As a result, the glycosylated pembrolizumab Fc variants EC, FG, ECFG, SPEC, SPFG, and SPECFG of the present disclosure had higher degradation temperatures than that of wild-type IgG4, and the conventional S228P/L235E variant SPLE showed a decreased degradation temperature (FIG. 17).

### Example 20. Analysis of Binding Affinity for Animal Fc Receptors of Glycosylated Pembrolizumab Fc Variants

### 20-1. Expression and Purification of Murine and Cynomolgus Monkey FcγRs

For analysis of the binding affinity of the glycosylated Fc variants of the present disclosure for murine and cynomolgus monkey FcγRs, which were widely used as preclinical animal models, murine FcγRI-GST, murine FcγRIIb-GST, murine FcγRIII-GST, murine FcγRIV-GST, cynomolgus monkey FcγRI-GST, cynomolgus monkey FcγRIIa-GST, cynomolgus monkey FcγRIIb-GST, and cynomolgus monkey FcγRIII-GST were produced as Fc receptor proteins of each animal. Specifically, the respective receptor proteins were cloned and prepared into an animal cell expression vector, transfected into Expi293F cells using PEI, and then incubated for 7 days under conditions of 37° C., 125 rpm, and 8% CO₂. After incubation, the supernatant was collected, equilibrated with PBS, and purified by anti-GST affinity chromatography.

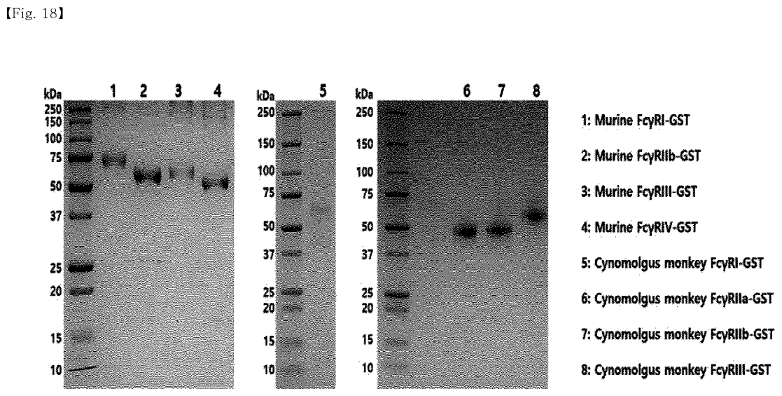

As a result, it was confirmed that murine FcγRI-GST, murine FcγRIIb-GST, murine FcγRIII-GST, murine FcγRIV-GST, cynomolgus monkey FcγRI-GST, cynomolgus monkey FcγRIIa-GST, cynomolgus monkey FcγRIIb-GST, and cynomolgus monkey FcγRIII-GST were purified with high purity (FIG. 18).

### 20-2. Analysis of Binding Affinity for Murine FcγRs of Glycosylated Pembrolizumab Fc Variants

To confirm the binding affinity for murine FcγRs of the glycosylated pembrolizumab Fc variants of the present disclosure purified in Example 15 above, ELISA analysis was performed. Specifically, 50 μl of murine FcγRI-GST, murine FcγRIIb-GST, murine FcγRIII-GST, and murine FcγRIV-GST diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hours. The plate was washed four times with 180 μl of 0.05% PBST, and then 50 μl of the glycosylated pembrolizumab Fc variants EC, FG, ECFG, SPEC, SPFG and SPECFG serially diluted with 1% skim milk were dispensed into each well and reacted at room temperature for 1 hour. After washing, antibody reaction was performed for 1 hour at room temperature using 50 μl of HRP-Protein L (GenScript, M00098) and then washed again. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

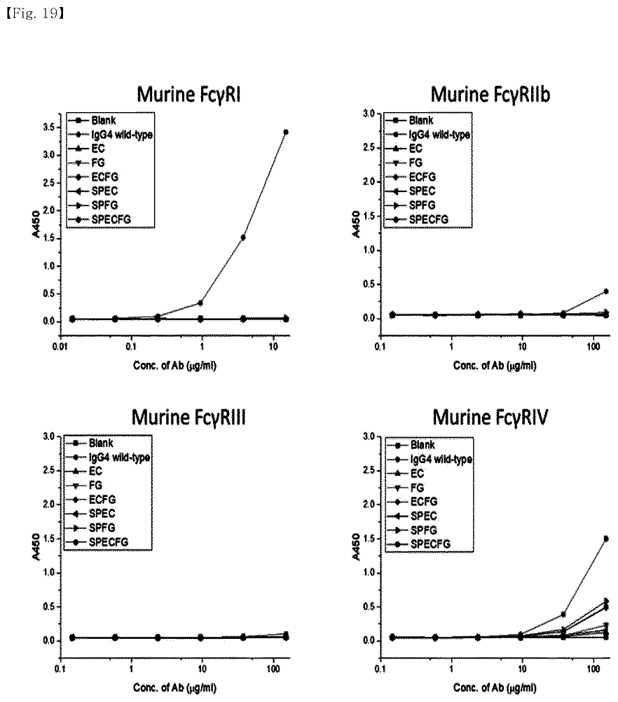

As a result, it was shown that all of the glycosylated pembrolizumab Fc variants discovered in the present disclosure had significantly lower FcγRs binding affinity than the wild-type IgG4 antibody, and in particular, the binding affinity for murine FcγRI, murine FcγRIIb, and murine FcγRIII was completely removed (FIG. 19).

### 20-3. Analysis of Binding Affinity for Monkey FcγRs of Glycosylated Pembrolizumab Fc Variants

To confirm the binding affinity for cynomolgus monkey FcγRs of the glycosylated pembrolizumab Fc variants of the present disclosure purified in Example above, ELISA analysis was performed. Specifically, 50 μl of cynomolgus monkey FcγRI-GST, cynomolgus monkey FcγRIIa-GST, cynomolgus monkey FcγRIIb-GST, and cynomolgus monkey FcγRIII-GST diluted to 4 μg/ml in 0.05 M Na₂CO₃ (pH 9.6) were immobilized on a flat bottom polystyrene high bind 96-well microplate (Costar, 3590) for 16 hours at 4° C., respectively, and then blocked with 100 μl of 4% skim milk (GenomicBase, SKI400) at room temperature for 1 hour. The plate was washed four times with 180 μl of 0.05% PBST, and then 50 μl of the glycosylated pembrolizumab Fc variants EC, FG, ECFG, SPEC, SPFG and SPECFG serially diluted with 1% skim milk were dispensed into each well and reacted at room temperature for 1 hour. After washing, antibody reaction was performed for 1 hour at room temperature using 50 μl of HRP-Protein L (GenScript, M00098), and then washed again. 50 μl of a 1-Step Ultra TMB-ELISA substrate solution (Thermo Fisher Scientific, 34028) was added to develop color, and then added with 50 μl each of 2 M H₂SO₄ to terminate the reaction. Absorbance was analyzed using an Epoch microplate spectrophotometer (BioTek).

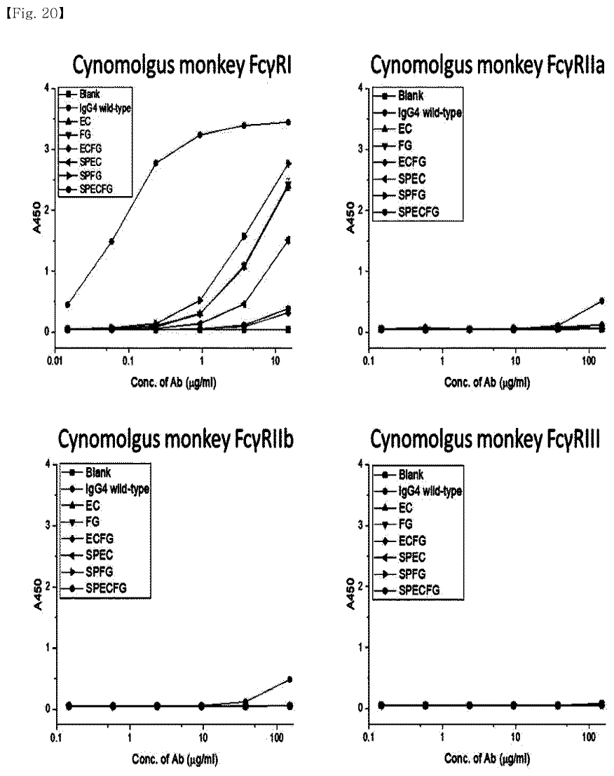

As a result, it was shown that all of the glycosylated pembrolizumab Fc variants discovered in the present disclosure had significantly lower FcγRs binding affinity than the wild-type IgG4 antibody (FIG. 20).

1. A human antibody Fc domain variant in which one or more amino acids selected from the group consisting of amino acids at positions 228, 233, 234, 291, 309 and 402 numbered according to a Kabat numbering system in a wide-type human antibody Fc domain are substituted with sequences different from wild-type amino acids.

2. The human antibody Fc domain variant of claim 1, wherein the human antibody Fc domain variant includes one or more amino acid substitutions selected from the group consisting of S228P, E233C, E233P, E233G, F234G, F234T, F234R, P291S, L309P and G402D.

3. The human antibody Fc domain variant of claim 1, wherein the human antibody Fc domain variant includes an amino acid substitution of E233C or F234G.

4. The human antibody Fc domain variant of claim 1, wherein the human antibody Fc domain variant includes amino acid substitutions of S228P and E233C.

5. The human antibody Fc domain variant of claim 1, wherein the human antibody Fc domain variant includes amino acid substitutions of S228P and F234G.

6. The human antibody Fc domain variant of claim 1, wherein the human antibody Fc domain variant includes amino acid substitutions of E233C and F234G.

7. The human antibody Fc domain variant of claim 1, wherein the human antibody Fc domain variant includes amino acid substitutions of E233G and L309P.

8. The human antibody Fc domain variant of claim 1, wherein the human antibody Fc domain variant includes amino acid substitutions of F234G and G402D.

9. The human antibody Fc domain variant of claim 1, wherein the human antibody Fc domain variant includes amino acid substitutions of E233P and P291S.

10. The human antibody Fc domain variant of claim 1, wherein the human antibody Fc domain variant includes amino acid substitutions of S228P, E233C and F234G.

11. The human antibody Fc domain variant of claim 1, wherein the human antibody is IgG4.

12. The human antibody Fc domain variant of claim 1, wherein the binding affinity for Fc gamma receptors (FcγRs) or C1q is reduced compared to a wild-type human antibody Fc domain.

13. The human antibody Fc domain variant of claim 1, wherein an effector function is reduced compared to the wild-type human antibody Fc domain.

14. The human antibody Fc domain variant of claim 13, wherein the effector function is an Fc-mediated effector function selected from C1q-binding, complement activation, complement dependent cytotoxicity (CDC), antibody-dependent cellular cytotoxicity (ADCC), Fc-receptor binding including Fc-gamma receptor binding, protein A-binding, protein G-binding, antibody-dependent cell-mediated phagocytosis (ADCP), complement dependent cell-mediated cytotoxicity (CDCC), complement-enhanced cytotoxicity, opsonization, Fc-containing polypeptide internalization, target downmodulation, ADC uptake, induction of apoptosis, cell death, cell cycle arrest, and any combination thereof.

15. An antibody or an immunologically active fragment thereof, comprising the Fc domain variant of claim 1.

16. The antibody or immunologically active fragment thereof of claim 15, wherein the binding affinity for Fc gamma receptors (FcγRs) or C1q is reduced compared to a wild-type human antibody.

17. The antibody or immunologically active fragment thereof of claim 15, wherein an effector function is reduced compared to the wild-type human antibody.

18. A nucleic acid molecule encoding the human antibody Fc domain variant of claim 1, or an antibody or immunologically active fragment thereof comprising the human antibody Fc domain variant.

19. An antibody therapeutic agent in which the antibody or immunologically active fragment thereof of claim 15 is conjugated with one or more therapeutic agents.

20. The antibody therapeutic agent of claim 19, wherein the antibody therapeutic agent has a reduced effector function.

21. The antibody therapeutic agent of claim 19, wherein the therapeutic agent is selected from a chimeric antigen receptor (CAR) cell therapy, an oncolytic drug, an immunotherapy agent, a cytotoxic agent, an angiogenesis inhibitor, a kinase inhibitor, a costimulatory molecule blocker, an adhesion molecule blocker, an anti-cytokine agent, an anti-CTLA-4 agent, an anti-PD-1 agent, an anti-PD-L1 agent, an anti-PD-L2 agent, a TNF-α cross-linking agent, a TRAIL cross-linking agent, an anti-CD27 agent, an anti-CD30 agent, an anti-CD40 agent, an anti-4-1BB agent, an anti-GITR agent, an anti-OX40 agent, an anti-TRAILR1 agent, an anti-TRAILR2 agent, tagretin, interferon-alpha, clobetasol, peginterferon, prednisone, romidepsin, bexarotene, methotrexate, triamcinolone cream, anti-chemokine, vorinostat, Gabapentin, cyclosporine, rapamycin, FK506, a detectable marker or reporter, a TNF antagonist, an antirheumatic agent, a muscle relaxant, narcotic, a non-steroidal anti-inflammatory drug (NSAID), analgesic, anesthetic, sedative, local anesthetic, neuromuscular blocker, antibacterial, psoriasis therapeutic agent, corticosteroid, anabolic steroid, erythropoietin, immunization, immunoglobulin, immunosuppressant, growth hormone, hormone replacement drug, radiopharmaceutical, antidepressant, antipsychotic, stimulant, asthma drug, beta agonist, inhaled steroid, epinephrine or analogue thereof, cytokine, cytokine antagonist, PD-1 antagonist, adenosine A2AR antagonist, CD73 inhibitor, CTLA-4 inhibitor, TIM-3 inhibitor, LAG-3 inhibitor, anthracycline, or any combination thereof.

22. A pharmaceutical composition for preventing or treating cancer comprising the human antibody Fc domain variant of claim 1, an antibody or immunologically active fragment thereof of comprising the human antibody Fc domain variant, or an antibody therapeutic agent in which the antibody or immunologically active fragment thereof is conjugated with one or more therapeutic agents as an active ingredient.

23. A method for preparing a human antibody Fc domain variant comprising:
a) incubating a host cell comprising a vector including a nucleic acid molecule encoding the human antibody Fc domain variant of claim 1; and
b) recovering a polypeptide expressed by the host cell.

24. A method for preparing an antibody with a reduced effector function, comprising:
a) incubating a host cell comprising a vector including a nucleic acid molecule encoding the antibody or immunologically active fragment thereof of claim 15; and
b) purifying the antibody expressed by the host cell.

25. (canceled)

26. (canceled)

27. A method for treating cancer comprising administering the human antibody Fc domain variant of claim 1, an antibody or immunologically active fragment comprising the human antibody Fc domain variant, or an antibody therapeutic agent in which the antibody or immunologically active fragment thereof is conjugated with one or more therapeutic agents in a pharmaceutically effective amount, to a subject with cancer.


SEQUENCE LISTING (30 sequences)

SEQ ID NO: 1 - protein, 229 aa, synthetic construct
ESKYGPPCPPCPAPCFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 2 - DNA, 687 bp, synthetic construct
  source 1..687: mol_type = other DNA
gagagcaagtacggaccaccctgtcctccctgtcctgctccttgtttcctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 3 - protein, 229 aa, synthetic construct
ESKYGPPCPPCPAPEGLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 4 - DNA, 687 bp, synthetic construct
  source 1..687: mol_type = other DNA
gagagcaagtacggaccaccctgtcctccctgtcctgctcctgaggggctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 5 - protein, 229 aa, synthetic construct
ESKYGPPCPSCPAPCGLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 6 - DNA, 687 bp, synthetic construct
  source 1..687: mol_type = other DNA
gagagcaagtacggaccaccctgtccttcatgtcctgctccttgtgggctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 7 - protein, 229 aa, synthetic construct
ESKYGPPCPPCPAPCGLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 8 - DNA, 687 bp, synthetic construct
  source 1..687: mol_type = other DNA
gagagcaagtacggaccaccctgtcctccctgtcctgctccttgtgggctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 9 - protein, 229 aa, synthetic construct
ESKYGPPCPSCPAPCFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 10 - DNA, 687 bp, synthetic construct
  source 1..687: mol_type = other DNA
gagagcaagtacggaccaccctgtccttcatgtcctgctccttgtttcctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 11 - protein, 229 aa, synthetic construct
ESKYGPPCPSCPAPEGLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 12 - DNA, 687 bp, synthetic construct
  source 1..687: mol_type = other DNA
gagagcaagtacggaccaccctgtccttcatgtcctgctcctgaggggctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 13 - protein, 229 aa, Homo sapiens
ESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - DNA, 687 bp, Homo sapiens
  source 1..687: mol_type = genomic DNA
gagagcaagtacggaccaccctgtccttcatgtcctgctcctgagttcctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 15 - protein, 229 aa, synthetic construct
ESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 16 - DNA, 687 bp, synthetic construct
  source 1..687: mol_type = other DNA
gagagcaagtacggaccaccctgtcctccctgtcctgctcctgagttcctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 17 - protein, 229 aa, synthetic construct
ESKYGPPCPPCPAPEFEGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 18 - DNA, 687 bp, synthetic construct
  source 1..687: mol_type = other DNA
gagagcaagtacggaccaccctgtcctccctgtcctgctcctgagttcgagggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 19 - protein, 229 aa, synthetic construct
ESKYGPPCPSCPAPPFLGGPSVFLFPPKPKDTLMISRTPEVTCVVVDVSQEDPEVQFNWYVDGVEVHNAKTKSREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - DNA, 687 bp, synthetic construct
  source 1..687: mol_type = other DNA
gagagcaagtacggaccaccctgtccttcatgtcctgctcctccgttcctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagtctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 21 - protein, 229 aa, synthetic construct
ESKYGPPCPSCPAPGFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - DNA, 687 bp, synthetic construct
  source 1..687: mol_type = other DNA
gagagcaagtacggaccaccctgtccttcatgtcctgctcctgggttcctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 23 - protein, 229 aa, synthetic construct
ESKYGPPCPSCPAPGFLGGPSVFLFPPKPKDTLMISRTPEVTCVVVDVSQEDPEVQFNWYVDGVEVHNAKTKPREEQFNSTYRVVSVLTVPHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - DNA, 687 bp, synthetic construct
  source 1..687: mol_type = other DNA
gagagcaagtacggaccaccctgtccttcatgtcctgctcctgggttcctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgccgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 25 - protein, 229 aa, synthetic construct
ESKYGPPCPSCPAPEGLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDDSFFLYSRLTVDKSRWQEGNVFSCSVMHEALHNHYTQKSLSLSLGK

SEQ ID NO: 26 - DNA, 687 bp, synthetic construct
  source 1..687: mol_type = other DNA
gagagcaagtacggaccaccctgtccttcatgtcctgctcctgagggtctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatgactctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 27 - protein, 229 aa, synthetic construct
ESKYGPPCPSCPAPETLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - DNA, 687 bp, synthetic construct
  source 1..687: mol_type = other DNA
gagagcaagtacggaccaccctgtccttcatgtcctgctcctgagacgctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag

SEQ ID NO: 29 - protein, 229 aa, synthetic construct
ESKYGPPCPSCPAPERLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 30 - DNA, 687 bp, synthetic construct
  source 1..687: mol_type = other DNA
gagagcaagtacggaccaccctgtccttcatgtcctgctcctgagcggctgggcgggccttccgtgttcctgtttcctccaaagccaaaagataccctgatgatctctcggaccccagaggtgacatgcgtggtggtggacgtgagccaggaggatcccgaggtgcagttcaactggtacgtggacggcgtggaggtgcacaatgccaagaccaagcctagggaggagcagtttaacagcacctaccgcgtggtgtccgtgctgacagtgctgcaccaggattggctgaacggcaaggagtataagtgcaaggtgtccaataagggcctgcccagctccatcgagaagaccatctctaaggccaagggccagcctagggagccacaggtgtacacactgccccctagccaggaggagatgaccaagaaccaggtgtccctgacatgtctggtgaagggcttctatccctccgacatcgccgtggagtgggagtctaatggccagcctgagaacaattacaagaccacaccacccgtgctggactccgatggctctttctttctgtattctcggctgacagtggataagagcagatggcaggagggcaacgtgttcagctgtagcgtgatgcacgaagcactgcacaaccattacactcagaaaagcctgtcactgtccctgggcaag